(19) United States
(12) Patent Application Publication
(10) Pub. No.: US 2020/0148803 A1
(43) Pub. Date: May 14, 2020

(54) POLYMERIZABLE MULTIFUNCTIONAL ANTIMICROBIAL QUATERNARY AMMONIUM MONOMERS, METHODS OF SYNTHESIS, AND USES THEREOF

(71) Applicant: ADA Foundation, Chicago, IL (US)

(72) Inventors: Stanislav Frukhtbeyn, Montgomery Village, MD (US); Ugochukwu Okeke, Upper Marlboro, MD (US); Diane Bienek, Gaithersburg, MD (US); Drago Skrtic, Gaithersburg, MD (US); Joseph Antonucci, Kenningston, MD (US); Anthony Giuseppetti, Frederick, MD (US)

(73) Assignee: ADA Foundation, Chicago, IL (US)

(21) Appl. No.: 16/673,408

(22) Filed: Nov. 4, 2019

Related U.S. Application Data
(60) Provisional application No. 62/757,781, filed on Nov. 9, 2018.

(51) Int. Cl. C08F 220/36; A01N 37/14; A61K 8/81

(52) U.S. Cl. CPC C08F 220/36; C08K 5/08; A61K 8/8152; A01N 37/14

(57) ABSTRACT

A polymerizable multifunctional antimicrobial monomer includes a quaternary ammonium cation group, wherein the quaternary ammonium cation ion group is bonded to functional groups and anions having structures chosen from the groups

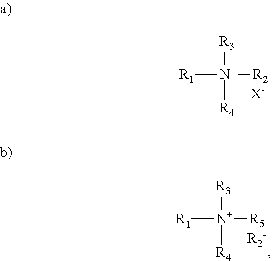

in which structure a) includes functional groups R₁, R₂, R₃, R₄ and anion X and structure b) includes functional groups R₁, R₃, R₄, R₅ and anion R₂, and in which structure a) is one of an ammonium monomer adhesive (AMadh) configuration and an ammonium monomer silane (AMsil) configuration, and structure b) is an ammonium monomer miscible (AMmis) configuration.

### RELATED APPLICATIONS

This application claim priority to provisional patent application 62/757,781, filed Nov. 9, 2018, entitled “POLYMERIZABLE MULTIFUNCTIONAL ANTIMICROBIAL QUATERNARY AMMONIUM MONOMERS, METHODS OF SYNTHESIS, AND USES THEREOF,” the content of which is incorporated herein by reference.

### STATEMENT OF FEDERAL GOVERNMENT SUPPORT

This application is supported in part by Grant DE026122 from the National Institute of Dental and Craniofacial Research (NIDCR). The Federal government has certain rights in this application.

### BACKGROUND

Current dental restorative materials do not possess substantial antimicrobial properties. Their major drawbacks arise from a compromised bonding integrity between the restoration and tooth enamel/dentin that eventually leads to compromised margin integrity, and bacterial infiltration of restoration to tooth interface, both of which lead to secondary caries and failure of the restoration. Most of the efforts to add antimicrobial function to dental materials have been focused on release of various low molecular weight compounds such as antibiotics, zinc, silver, fluoride, iodide ions or chlorhexidine. There are numerous unresolved issues related to the activities of these agents, such as short-lived release kinetics, compromised mechanical properties, toxicity to surrounding tissues, development of tolerances and questionable long-term effectiveness of their antimicrobial action.

### SUMMARY

Disclosed are polymerizable multifunctional antimicrobial monomers that contain a quaternary ammonium cation group that is bonded to groups R₁, R₂, R₃, R₄ and anion X or groups R₁, R₃, R₄, R₅ and anion R₂.

The following schematics show embodiments of the disclosed configurations, where schematic (a) illustrates ammonium monomer adhesive (AMadh) and ammonium monomer silane (AMsil) configurations and (b) illustrates ammonium monomer miscible (AMmis) configurations.

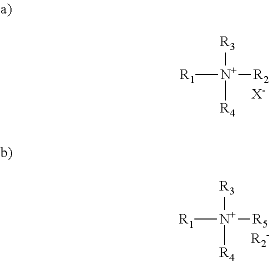

These novel antimicrobial monomers are designed to interact with copolymer resin matrix and tooth enamel or dentin structures through the added adhesive functionalities in monomer structure (AMadh series), couple with both filler phase and polymer phase of the composites (AMsil series), and act as polymerizable amphiphilic surfactants to facilitate monomer and filler miscibility (AMmis series).

In an embodiment of the AMadh series compounds, R₁ comprises an alkenyl group, which functions as a polymerization site between the quaternary ammonium monomer and copolymer or composite resins. Example alkenyl groups include vinyl (CH₂═CH—), methacrylic (CH₂═C(CH₃)—) and crotyl (CH₃—CH═CH—CH₂—) groups. The R₂ group comprises a carboxylic group to facilitate adhesion of the resin to mineralized tooth tissue (enamel or dentin). The X anion may be a halogen anion (F, Cl, Br, I).

In an embodiment of the AMsil series compounds, R₁ comprises an alkenyl group that functions as a polymerization site between quaternary ammonium monomers and copolymer or composite resins. Example alkenyl groups include vinyl (CH₂═CH—), methacrylic (CH₂═C(CH₃)—) and crotyl (CH₃—CH═CH—CH₂—) groups. The R₂ group comprises an organo-silane group which functions as a coupling agent to fillers and resin polymer matrix. The X anion is a halogen anion (F, Cl, Br, I).

In an embodiment of the AMmis compounds, AMmis1, R₁ comprises an alkenyl group that functions as a polymerization site between quaternary ammonium monomers and copolymer or composite resins. Example alkenyl groups include vinyl (CH₂═CH—), methacrylic (CH₂═C(CH₃)—) and crotyl (CH₃—CH═CH—CH₂—) groups. The R₂ anion comprises an organo-sulfate group with a carbon tail to impart amphiphilic character to a quaternary ammonium compound, which will facilitate monomer and filler miscibility.

In another embodiment of the AMmis compounds, AMmis2, R₁ comprises an alkenyl group which functions as polymerization site between quaternary ammonium monomers and copolymer or composite resins. Example alkenyl groups include vinyl (CH₂═CH—), methacrylic (CH₂═C(CH₃)—) and crotyl (CH₃—CH═CH—CH₂—) groups. The R₂ anion comprises an ionic acrylate or methacrylate functionality, which also participates in polymerization reaction and facilitates monomer and filler miscibility.

In a synthesis embodiment, a polymerizable multifunctional quaternary ammonium compound may be produced through a Menschutkin reaction, where a tertiary amine reacts with an organo halide.

In another synthesis embodiment, a polymerizable multifunctional quaternary ammonium compound may be produced through an ion exchange reaction between a halogenated quaternary amine and metal organo-sulfate, where M=metal, R₂=organo-sulfate.

In yet another synthesis embodiment, a polymerizable multifunctional quaternary ammonium compound may be produced through ion exchange reaction between a halogenated quaternary amine and metal acrylate or methacrylate, where M=metal, R₂=ionic acrylate or methacrylate.

In an embodiment, the herein disclosed compositions may be mixed with medical/dental resins of choice and polymerized to form a copolymer. The resins may be composed of acrylic, methacrylic, and acrylic urethane compounds that are suitable for use in humans. In some embodiments, resins may be chosen from the group consisting of triethylene glycol dimethacrylate (TEGDMA), hydroxyethyl methacrylate (HEMA), ethyl hydroxymethyl acrylate (EHMA), bisphenol A-glycidyl methacrylate (bisGMA), urethane dimethacrylate (UDMA), polyethylene glycol extended urethane dimethacrylate (PEG-UDMA), or their mixtures. Fillers can also be incorporated into the resin formulations as reinforcing and/or remineralizing agents, thereby forming composite resins (also known as composites).

### DESCRIPTION OF THE DRAWINGS

The Detailed Description refers to the following Figures, in which like numbers, letters, and symbols refer to like items, and in which:

FIGS. 1-32 illustrate results of experiments to validate and characterize the synthesized quaternary ammonium halogen salt monomers and amphiphilic quaternary ammonium antimicrobial monomers;

FIGS. 33-35 illustrate the effectiveness of copolymer resins containing the herein disclosed antimicrobial monomers against planktonic and biofilm forms of Streptococcus mutans and Porphyromonas gingivalis;

FIGS. 36A-41 illustrate the biocompatibility of the herein disclosed antimicrobial monomers; and

FIG. 42 illustrates the effect of the herein disclosed antimicrobial monomers on shear bond strength of a multipurpose dental restorative material.

### DETAILED DESCRIPTION

Current dental restorative materials do not possess substantial antimicrobial properties. Their major drawbacks arise from a compromised bonding integrity between the restoration and tooth enamel/dentin that eventually leads to compromised margin integrity, bacterial infiltration of restoration to tooth interface which leads to secondary caries and failure of the restoration. Most efforts to add antimicrobial function to dental materials have focused on release of various low molecular weight compounds such as antibiotics, zinc, silver, fluoride, iodide ions, or chlorhexidine. There are numerous unresolved issues related to the activities of these agents, such as short-lived release kinetics, compromised mechanical properties, toxicity to surrounding tissues, development of tolerances, and questionable long-term effectiveness of their antimicrobial action. Recently, quaternary ammonium methacrylates have been utilized in dental materials and have been shown to be effective against Gram-positive and Gram-negative bacteria. See, generally, S. Imazato. Bioactive restorative materials with antibacterial effect: new dimension of innovation in restorative dentistry. Dent Mater J 2009; 28(1): 11-19, [PMID: 19280964]; E R Kenawy et al. The chemistry and application of antimicrobial polymers: A state-of-the-art review. Biomacromolecules 2002; 8(5): 1359-1384, [PMID: 17425365]; and F. Li et al. Effects of a dental adhesive incorporating antibacterial monomer on the growth, adherence and membrane integrity of Streptococcus mutans. J Dent 2009; 37: 289-296 [PMID: 19185408]. Most attention has been given to methacryloyloxydodecyl pyrimidinium bromide (MDBP) and the preparation of its acrylamide copolymer. See T. Thome et al. In vitro analysis of the antibacterial monomer MDPB-containing restorations on the progression of secondary caries. J Dent 2009; 37: 705-711, [PMID: 19540033]. The MDBP-containing primer has been commercialized and the MDBP has been suggested as potentially applicable to various restoratives. However, the composites with MDBP show poor color stability and their use is limited to the restorations of root cavities in areas where aesthetics is not an issue. Ionic dimethacrylates, disclosed in U.S. Pat. No. 8,217,081 are another group of quaternary ammonium salts that demonstrate antimicrobial activity and miscibility. However, they are only designed to interact with resin copolymers through their acrylic or methacrylic groups.

### COMPOSITION

Disclosed are polymerizable multifunctional antimicrobial (AM) monomers that contain a quaternary ammonium cation group that is bonded to groups R₁, R₂, R₃, R₄ and anion X or groups R₁, R₃, R₄, R₅ and anion R₂. The following schematics show embodiments of the disclosed configurations, where schematic (a) illustrates ammonium monomer adhesive (AMadh) and ammonium monomer silane (AMsil) configurations and (b) illustrates ammonium monomer miscible (AMmis) configurations.

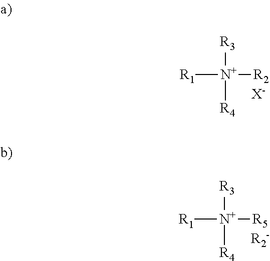

These novel antimicrobial monomers are designed to interact with copolymer resin matrix and tooth enamel or dentin structures through the added adhesive functionalities in monomer structure (AMadh series), couple with both filler phase and polymer phase of the composites (AMsil series), and act as polymerizable amphiphilic surfactants to facilitate monomer and filler miscibility (AMmis series).

In an embodiment of the AMadh series compounds, R₁ comprises an alkenyl group, which functions as polymerization site between quaternary ammonium monomers and copolymer or composite resins. Example alkenyl groups include vinyl (CH₂═CH—), methacrylic (CH₂═C(CH₃)—) and crotyl (CH₃—CH═CH—CH₂—) groups. The R₂ group comprises a carboxylic group to facilitate adhesion of the resin to mineralized tooth tissue (enamel or dentin). The anion X is a halogen anion (F, Cl, Br, I).

In an embodiment of the AMsil series compounds, R₁ comprises an alkenyl group that functions as polymerization site between quaternary ammonium monomers and copolymer or composite resins. Example alkenyl groups include vinyl (CH₂═CH—), methacrylic (CH₂═C(CH₃)—) and crotyl (CH₃—CH═CH—CH₂—) groups. The R₂ group comprises an organo-silane group that functions as a coupling agent to fillers and resin polymer matrix. The anion X is a halogen anion (F, Cl, Br, I).

In an embodiment of the AMmis compounds, AMmis1, R₁ comprises an alkenyl group that functions as a polymerization site between quaternary ammonium monomers and copolymer or composite resins. Example alkenyl groups include vinyl (CH₂═CH—), methacrylic (CH₂═C(CH₃)—) and crotyl (CH₃—CH═CH—CH₂—) groups. The R₂ anion comprises an organo-sulfate group with a carbon tail to impart amphiphilic character to a quaternary ammonium compound, which will facilitate monomer and filler miscibility.

In another embodiment of the AMmis compounds, AMmis2, R₁ comprises an alkenyl group which functions as polymerization site between quaternary ammonium monomers and copolymer or composite resins. Example alkenyl groups include vinyl (CH₂═CH—), methacrylic (CH₂═C(CH₃)—) and crotyl (CH₃—CH═CH—CH₂—) groups. The anion R₂ comprises an ionic acrylate or methacrylate functional group, which also participates in polymerization reaction and facilitates monomer and filler miscibility.

In a synthesis embodiment, a polymerizable multifunctional quaternary ammonium compound may be produced through a Menschutkin reaction, where a tertiary amine reacts with an organo halide. Equation 1 below represents a generic form of this reaction:

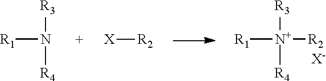

In another synthesis embodiment, shown in Equation 2 below, a polymerizable multifunctional quaternary ammonium compound may be produced through an ion exchange reaction between a halogenated quaternary amine and metal organo-sulfate, where M=metal, R₂=organo-sulfate.

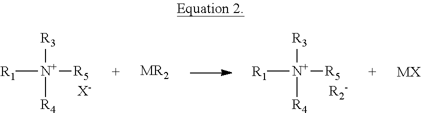

In another synthesis embodiment, shown in Equation 3 below, a polymerizable multifunctional quaternary ammonium compound may be produced through ion exchange reaction between a halogenated quaternary amine and metal acrylate or methacrylate, where M=metal, R₂=ionic acrylate or methacrylate.

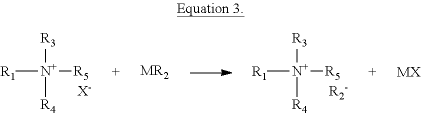

The herein disclosed compositions may be mixed with medical/dental resins of choice and polymerized to form a copolymer. For example, the resins may be composed of acrylic, methacrylic, and acrylic urethane compounds that are suitable for use in humans. In some examples, resins may be chosen from the group consisting of triethylene glycol dimethacrylate (TEGDMA), hydroxyethyl methacrylate (HEMA), ethyl hydroxymethyl acrylate (EHMA), bisphenol A-glycidyl methacrylate (bisGMA), urethane dimethacrylate (UDMA), polyethylene glycol extended urethane dimethacrylate (PEG-UDMA), or their mixtures. Fillers can also be incorporated into the resin formulations as reinforcing and/or remineralizing agents, thereby forming composite resins (also known as composites).

Resins may include 10-90 wt/wt % of the base monomer, for example bisGMA, UDMA, and PEG-UDMA, and 10-90 wt/wt % of the diluent monomer, for example TEGDMA, HEMA, and EHMA, which can be mixed with the polymerizable multifunctional antimicrobial compositions disclosed herein to form a mixed composition. The mixed composition may further include additives, fillers, and polymerization initiators such as for example, the initiators may be camphorquinone (CQ) and ethyl 4-N,N-dimethylaminobenzoate (4E). Exposure of the mixed compositions containing CQ and 4E to a light source will initiate polymerization. Other types of initiators may be used as well.

In other embodiments, the mixture may contain up to 50 wt/wt % of the polymerizable multifunctional antimicrobial compositions. The amount used may be chosen based on the desired effectiveness of the antimicrobial formulation and resin composition.

The compositions disclosed herein employ solvents and reagents procured from commercial sources and that were used without any further purification: 2-(dimethylamino)ethyl methacrylate (DMAEMA), 6-bromohexanoic acid (BrHA), 11-bromoundecanoic acid (BrUDA), butylated hydroxytoluene (BHT), and (3-iodopropyl)trimethoxysilane (IPTMS), chloroform, acetone, (11-bromoundecyl)trimethoxysilane (BrUDTMS), 2-(methacryloyloxy)-N,N,N-trimethylethan-1-aminium chloride (METMA-Cl), sodium dodecyl sulfate (SDS), sodium methacrylate (SMA).

### SYNTHESIS REACTIONS

Quaternary ammonium salts were synthesized by reacting a tertiary amine with alkyl halides, containing carboxylic (AMadh series) or organosilane (AMsil series) functional groups. Reaction between a neutral tertiary amine and an alkyl halide produces two ions of opposite sign, also known as a Menschutkin reaction, is a bimolecular nucleophilic substitution reaction.

### General Procedure for the Synthesis of Quaternary Ammonium Halogen Salt Monomers:

DMAEMA (10.0 mmol) was reacted with alkyl halide (10.0 mmol) at 50-55° C. in the presence of BHT (1.0 mmol) and chloroform (2.5 mL). Table 1 lists combinations of reacting compounds used in the herein disclosed processes to synthesize quaternary ammonium halogen salt monomers, and their corresponding reaction products. After 24 hours, the reaction products were collected, purified and dried as indicated below.

TABLE 1
Combinations of Reacting Compounds and Their Corresponding Reaction Products

| Tertiary amine | Alkyl halide |
|---|---|
| 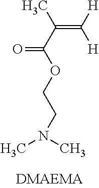 | 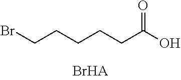 |
| | 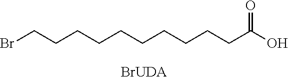 |
| | 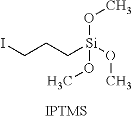 |
| | 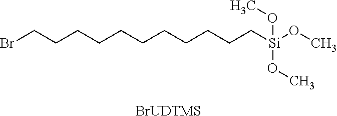 [SMILES: CO[Si](CCCCCCCCCCCBr)(OC)OC ; "BrUDTMS"] |

Product

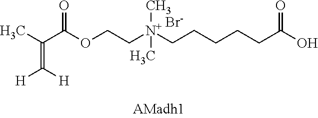 [SMILES: C=C(C)C(=O)OCC[N+](C)(C)CCCCCC(=O)O ; [Br-]]

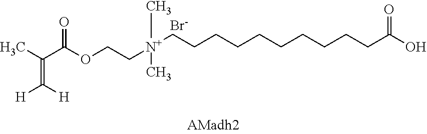 [SMILES: C=C(C)C(=O)OCC[N+](C)(C)CCCCCCCCCCC(=O)O ; [Br-]]

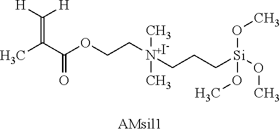 [SMILES: C=C(C)C(=O)OCC[N+](C)(C)CCC[Si](OC)(OC)OC ; [I-]]

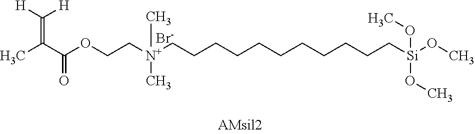 [SMILES: C=C(C)C(=O)OCC[N+](C)(C)CCCCCCCCCCC[Si](OC)(OC)OC ; [Br-]]

### AMadh Series Compounds (Reactions 1 and 2):

AMadh1 (5-carboxy-N-(2-(methacryloyloxy)ethyl)-N,N-dimethylpentan-1-aminium bromide Referring to Reaction 1, ethanol (2.0 mL) was added to the reaction mixture and transferred to a 50-mL conical separatory flask. The product was precipitated and washed with hexane (3×20 mL). The supernatant was discarded and acetone (40 mL) was used to re-dissolve the product. Hexane (10 mL) was added to this solution and AMadh1 precipitated as fine white powder. The product was collected by vacuum filtration, washed with hexane, and vacuum dried. The reaction yielded 2.4250 grams of purified product (68.8% yield).

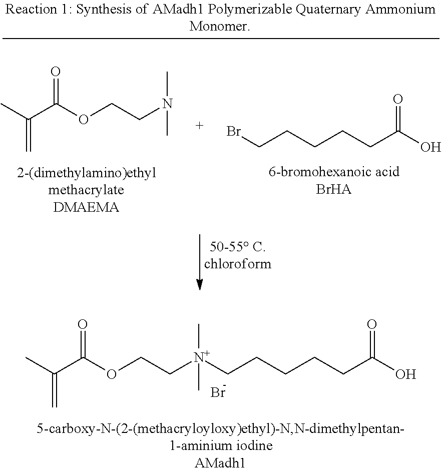 [SMILES: C=C(C)C(=O)OCCN(C)C ; O=C(O)CCCCCBr ; C=C(C)C(=O)OCC[N+](C)(C)CCCCCC(=O)O ; Br]

AMadh2 (10-carboxy-N-(2-(methacryloyloxy)ethyl)-N,N-dimethyldecan-1-aminium bromide. Referring to Reaction 2, the product precipitated out of solution as white powder. AMadh2 was collected by vacuum filtration and washed with chloroform. The reaction yielded 2.0983 grams of purified product (53.2% yield).

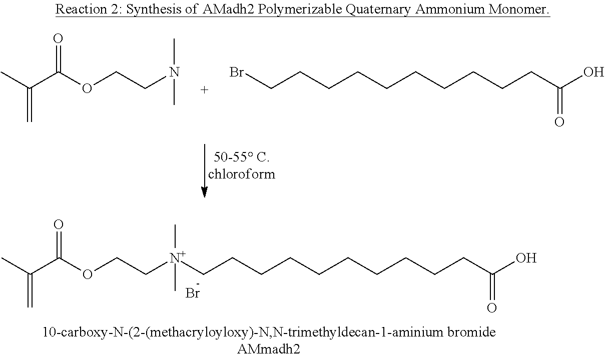 [SMILES: C=C(C)C(=O)OCCN(C)C ; O=C(O)CCCCCCCCCCBr ; C=C(C)C(=O)OCC[N+](C)(C)CCCCCCCCCCC(=O)O ; Br]

### AMsil Series Compounds (Reactions 3 and 4):

AMsil1 (N-(2-(methacryloyloxy)ethyl)-N,N-dimethyl-3-(trimethoxysilyl)propan-1-aminium iodide). Chloroform (5.0 mL) was used to rinse and transfer the reaction mixture to a beaker. Diethyl ether (20 mL) was used to precipitate the final product which was collected by vacuum filtration. AMsil1 was washed with hexane and vacuum dried. The reaction yielded 4.2420 grams of purified product (94.8% yield).

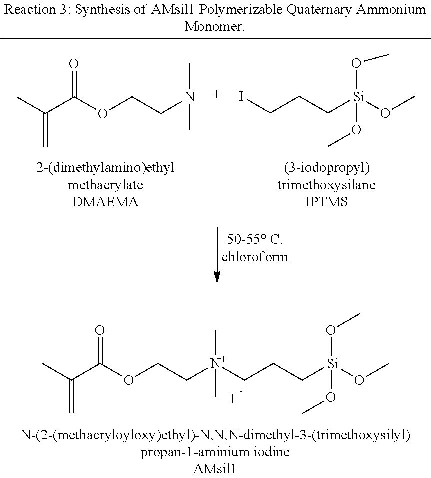

AMsil2 (N-(2-(methacryloyloxy)ethyl)-N,N-dimethyl-11-(trimethoxysilyl)undecan-1-aminium bromide). Diethyl ether (10 mL×3) was added to the reaction mixture to precipitate and wash the product. After each addition of diethyl ether, AMsil2 product was collected by centrifugation (14,000 rpm) and the supernatant top layer decanted. The AMsil2 product was vacuum dried. The reaction yielded 1.8474 grams of purified product (36.0% yield).

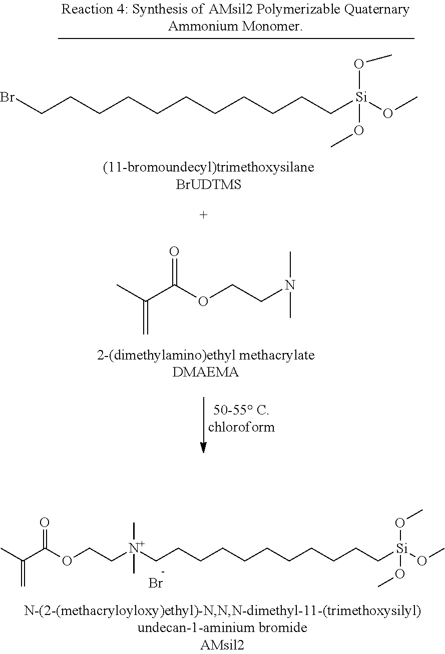

### General Procedure for the Synthesis of Amphiphilic Quaternary Ammonium Monomers:

AMmis1 synthesis. An aqueous solution of METMA-Cl, 80% w/w (10.0 mmol) was reacted with organo-sulfate containing a long carbon chain SDS (10.0 mmol) at 50-55° C. in the presence of ethanol (3.0 mL) and water (2.0 mL). After 24 hours, reaction products were collected, purified and dried as indicated below.

AMmis2 synthesis. An aqueous solution of METMA-Cl, 80% w/w (10.0 mmol) was reacted with ionic methacrylate SMA (10.0 mmol) at 50-55° C. in the presence of acetone (10.0 mL) and water (1.0 mL). After 24 hours, reaction products were collected, purified and dried as indicated below.

### AMmis Series Compounds (Reactions 5 and 6):

AMmis1 (2-(methacryloyloxy)-N,N,N-trimethylethan-1-aminium dodecyl sulfate). A reaction mixture was washed with hexane (3×5.0 mL) and the bottom layer containing the product was collected. Addition of excess acetone precipitated unreacted precursors which were filtered out. Supernatant was collected and dried in a rotary evaporator producing white powder product. The reaction yielded 1.9892 g of purified product (45.5% yield).

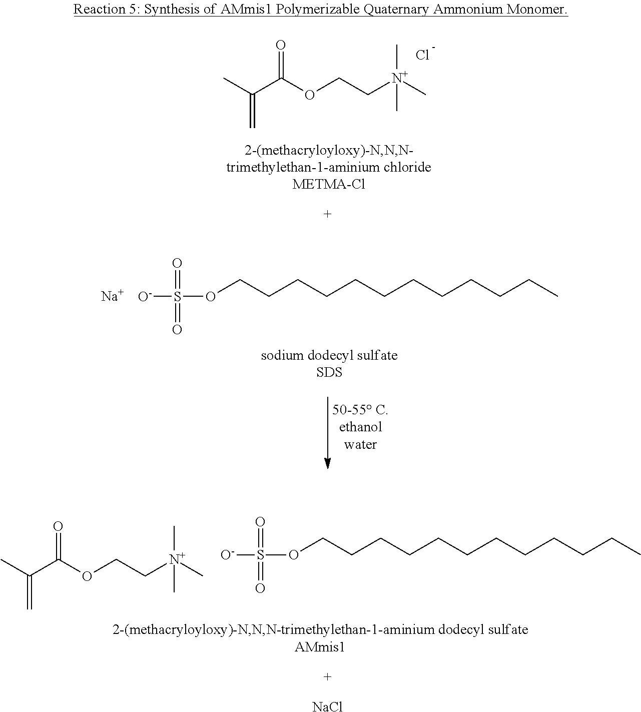

AMmis2 (2-(methacryloyloxy)-N,N,N-trimethylethan-1-aminium methacrylate). Unreacted precursors were precipitated with 1.0 mL of acetone and filtered out. Supernatant was collected and dried in a rotary evaporator producing a white powder product. The reaction yielded 2.1523 g of purified product (86.9% yield).

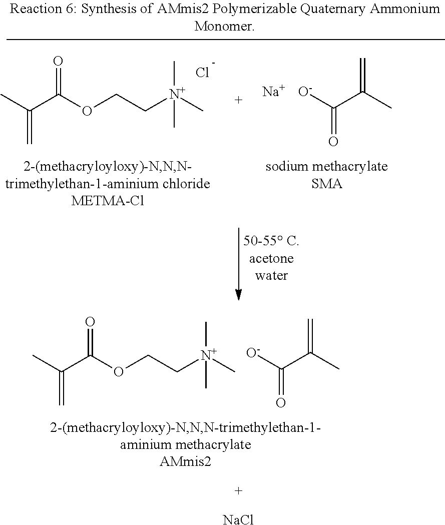

At room temperature and atmospheric pressure, quaternary ammonium compounds described in this disclosure are solids. They exhibit slow dissolution and miscibility with resin copolymer mixtures. This limitation can be overcome by dissolving quaternary ammonium compounds in volatile solvents (some of the examples are alcohols, chloroform, etc.). These solutions may be added to the copolymer resins and mixed to produce homogeneous copolymer mixtures. At this point, the mixtures can be placed into a vacuum chamber or gases (some of the examples are air, nitrogen, argon, etc.) may be passed over or through the mixtures resulting in removal of volatile solvents and production of polymerizable copolymer resins free of the solvent used.

### EXPERIMENTS AND EXPERIMENTAL RESULTS

Applicants conducted five series of experiments: a first experimental series validated and characterized the synthesized quaternary ammonium halogen salt monomers and amphiphilic quaternary ammonium antimicrobial monomers; a second experimental series to determine the physiochemical and mechanical properties of copolymer resins containing the herein disclosed antimicrobial monomers; a third experimental series tested the effectiveness of copolymer resins containing the herein disclosed antimicrobial monomers against planktonic and biofilm forms of Streptococcus mutans, and against biofilm forms of Porphyromonas gingivalis; a fourth experimental series tested the biocompatibility of the herein disclosed antimicrobial monomers; and a fifth experimental series tested the effect of the herein disclosed antimicrobial monomers on shear bond strength of a multipurpose dental restorative material.

### Experimental Series 1: Validation and Characterization of Synthesized Quaternary Ammonium Halogen Salt Monomers and Amphiphilic Quaternary Ammonium Monomers

### Materials and Methods:

Nuclear magnetic resonance spectra (NMR, 1H, 13C, Heteronuclear single quantum coherence spectroscopy, HSQC (2D: 1H-13C)) data of synthesized compounds was collected on Bruker Avance II 600 MHz spectrometer (Billerica, Mass., USA) equipped with a BBO room temperature probe. Deuterated dimethyl sulfoxide (DMSO-d6) containing tetramethylsilane (TMS) was used as a solvent. Mass spectra were collected on Waters Quattro Micro mass spectrometer (Waters Corp., Milford, Mass., USA) equipped with an ESI probe and operated in the positive or negative ionization mode. Fourier transform infra-red spectra were collected on a diamond ATR cell attached to Nexus 670 (ThermoFisher, Madison, Wis., USA) spectrometer, equipped with a DTGS room temperature detector (FTIR-ATR).

### Results:

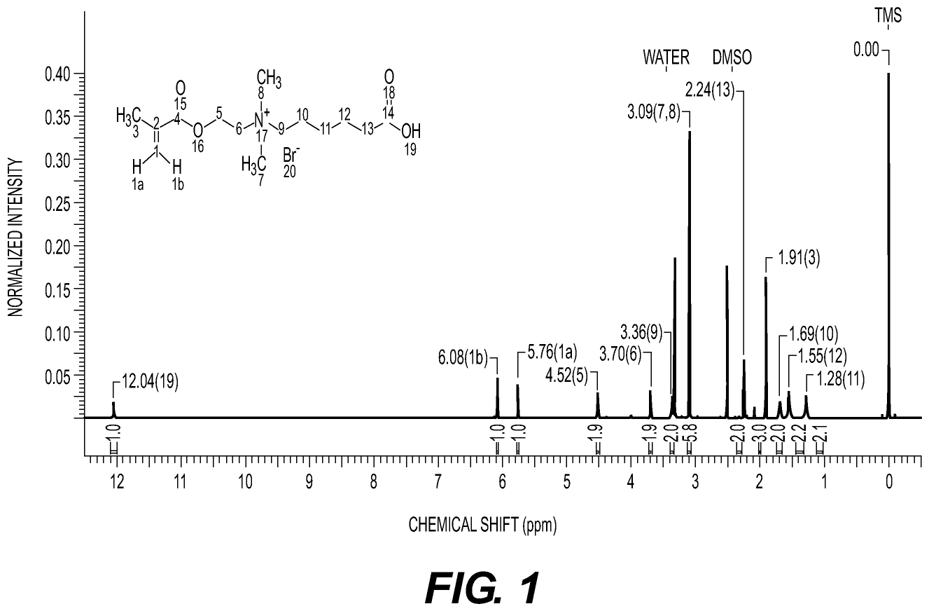

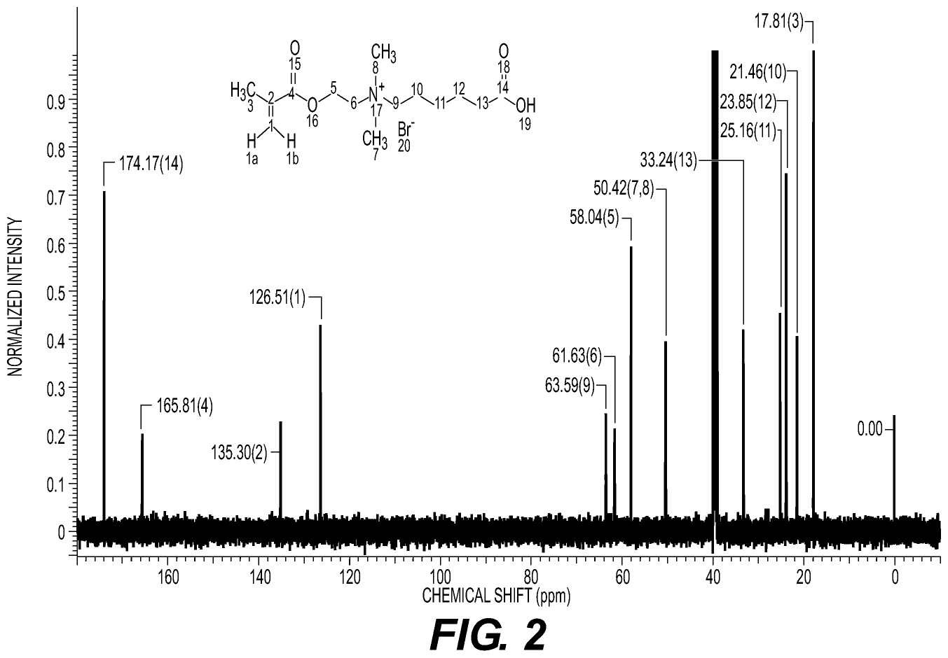

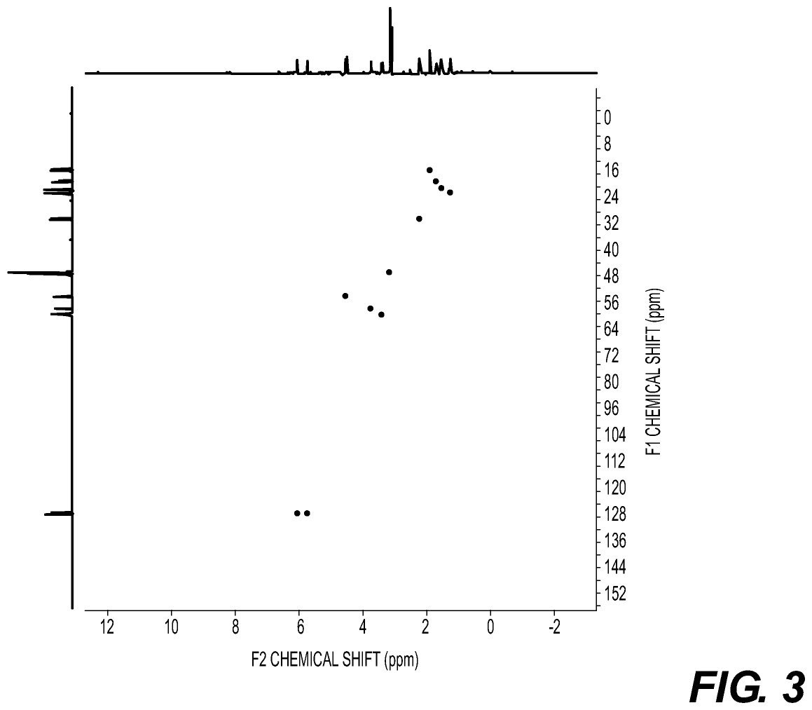

AMadh1 (5-carboxy-N-(2-(methacryloyloxy)ethyl)-N,N-dimethylpentan-1-aminium bromide). An AMadh1 compound was synthesized as described in the Reaction 1 schematic. FIG. 1 shows its proton (1H) NMR spectra, structure and proton (δH) chemical shifts, assignments and integration values. FIG. 2 shows its carbon (13C) NMR spectra, structure and carbon (δC) chemical shifts and assignments. FIG. 3 shows its HSQC spectra (1H chemical shifts are potted on X-axis(F2) and 13C chemical shifts on Y-axis(F1)). These results confirm successful quaternization of the tertiary amine reactant. Complete NMR structural assignment of AMadh1 compound is given in Table 2.

TABLE 2
NMR Spectroscopic Data for AMadh1.

| position | δC, ppm | δH, ppm |
|---|---|---|
| 1 | 126.5 | 5.76; 6.08 |
| 2 | 135.3 | |
| 3 | 17.8 | 1.91 |
| 4 | 165.8 | |
| 5 | 58.0 | 4.52 |
| 6 | 61.6 | 3.70 |
| 7, 8 | 50.4 | 3.09 |
| 9 | 63.6 | 3.36 |
| 10 | 21.5 | 1.69 |
| 11 | 25.2 | 1.28 |
| 12 | 23.9 | 1.55 |
| 13 | 33.2 | 2.24 |
| 14 | 174.2 | |
| 19 | | 12.04 |

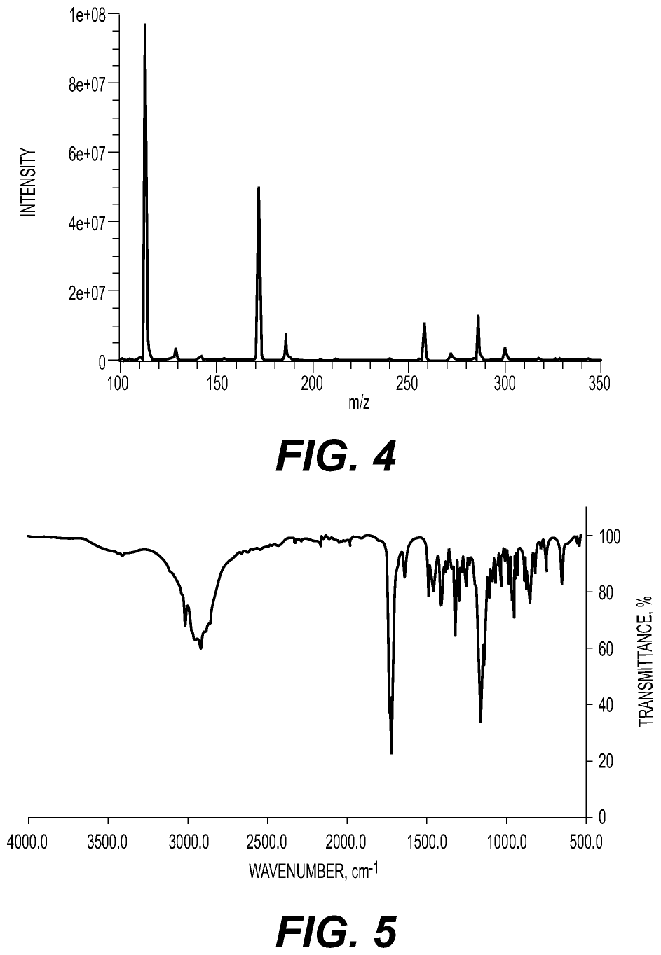

FIG. 4 shows the mass spectra of AMadh1 in the positive ionization mode (MS EI⁺). High abundance fragments, their mass-to-charge (m/z) ratio and corresponding structures observed were: MSEI⁺ m/z 113.0 (C₆H₉O₂., m/z=113.1), 114.1, 172.2 (C₉H₁₈NO₂.⁺, m/z=172.1), 186.3 (C₁₀H₂₀NO₂.⁺, m/z=186.1), 258.3 (C₁₃H₂₄NO₄².⁺, m/z=258.2), 286.3, 300.3.

FIG. 5 shows the FTIR-ATR spectra of AMadh1. Peak positions (νₘₐₓ) of the higher intensity peaks observed are: FTIR-ATR νₘₐₓ 3010.78, 2915.74, 1727.02, 1716.79, 1636.09, 1488.30, 1455.62, 1395.06, 1316.78, 1289.61, 1246.48, 1156.13, 1134.14 946.75 848.19, 814.90, 741.39, 645.12 cm⁻¹.

Collectively, FTIR, NMR and MS analysis of synthesized AMadh1 compound confirmed that its structure is as designed.

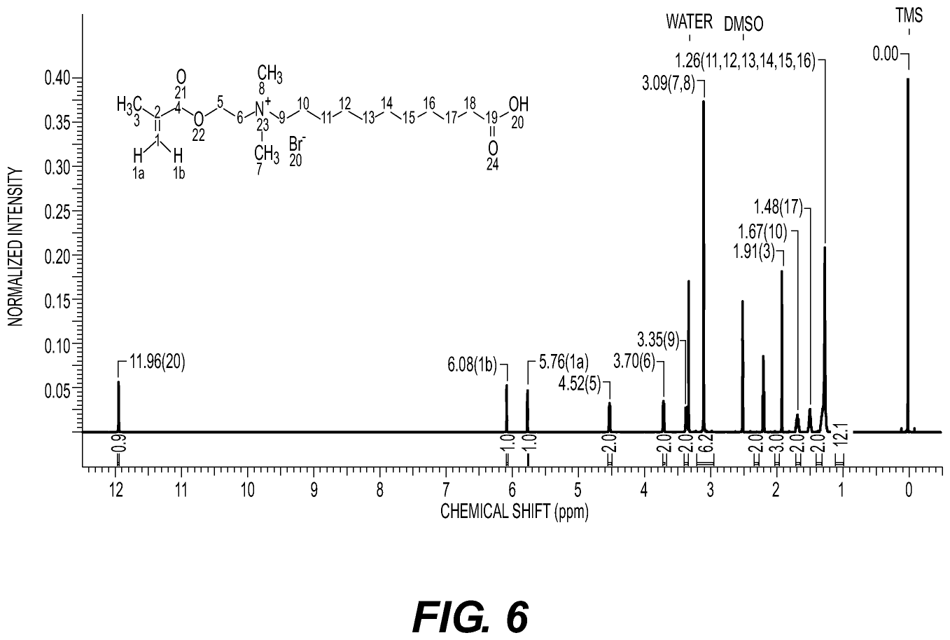

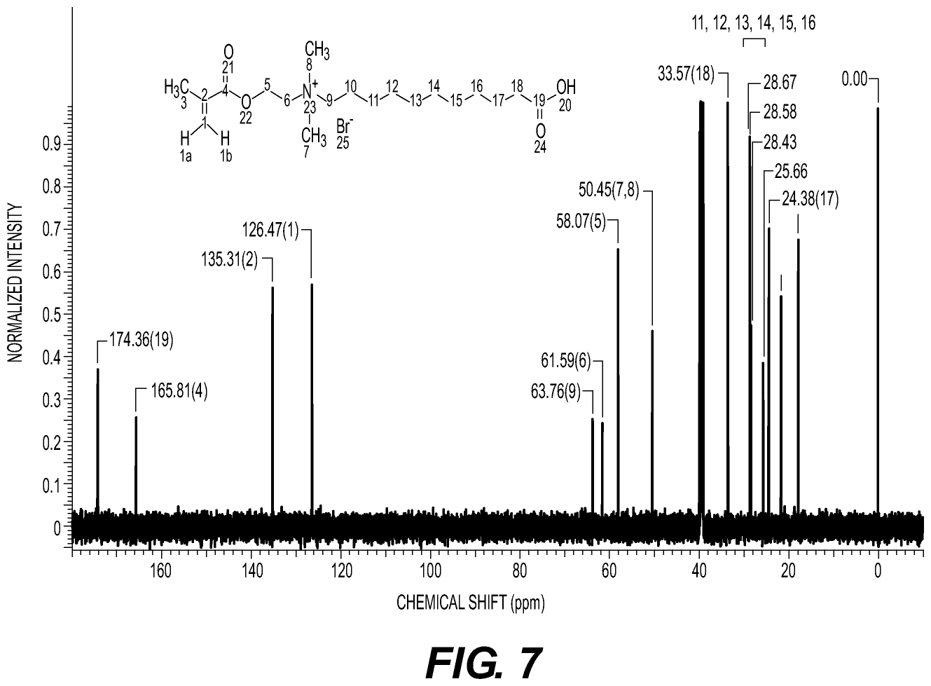

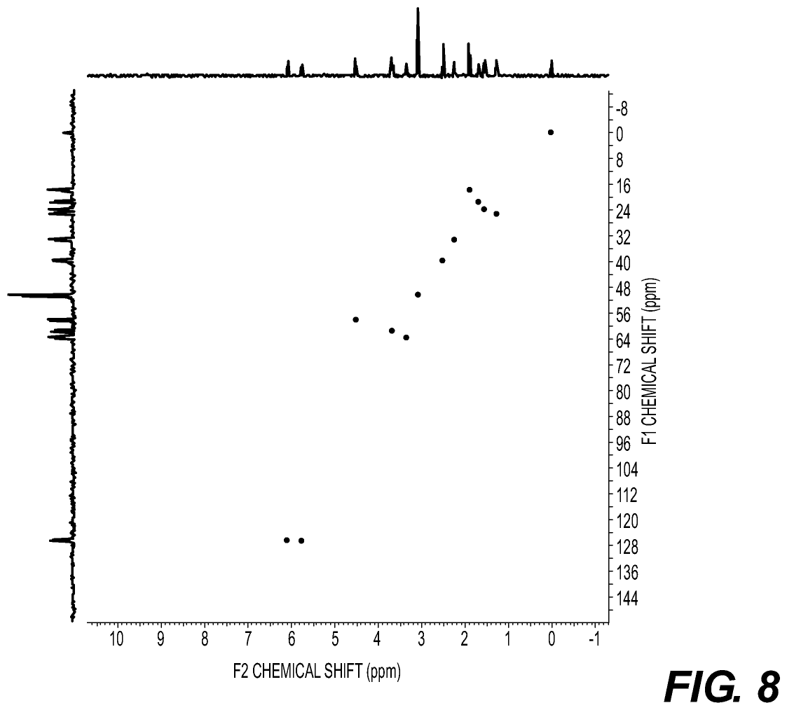

AMadh2 (10-carboxy-N-(2-(methacryloyloxy)ethyl)-N,N-dimethyldecan-1-aminium bromide). An AMadh2 compound was synthesized as described in the Reaction 2 schematic. FIG. 6 shows its proton (1H) NMR spectra, structure and proton (δH) chemical shifts, assignments and integration values. FIG. 7 shows its carbon (13C) NMR spectra, structure and carbon (δC) chemical shifts and assignments. FIG. 8 shows its HSQC spectra (1H chemical shifts are potted on X-axis(F2) and 13C chemical shifts on Y-axis(F1)). These results confirm successful quaternization of the tertiary amine reactant. Complete NMR structural assignment of AMadh2 compound is given in Table 3.

TABLE 3
NMR Spectroscopic Data for AMadh2.

| position | δC, ppm | δH, ppm |
|---|---|---|
| 1 | 126.5 | 5.76; 6.08 |
| 2 | 135.3 | |
| 3 | 17.8 | 1.91 |
| 4 | 165.8 | |
| 5 | 58.1 | 4.52 |
| 6 | 61.6 | 3.70 |
| 7, 8 | 50.4 | 3.09 |
| 9 | 63.8 | 3.35 |
| 10 | 21.7 | 1.67 |
| 11-16 | 25.7, 28.37, 28.43, 28.58, 28.64, | |
| | 28.67 | 1.26 |
| 17 | 24.4 | 1.48 |
| 18 | 33.6 | 2.19 |
| 19 | 174.4 | |
| 20 | | 11.96 |

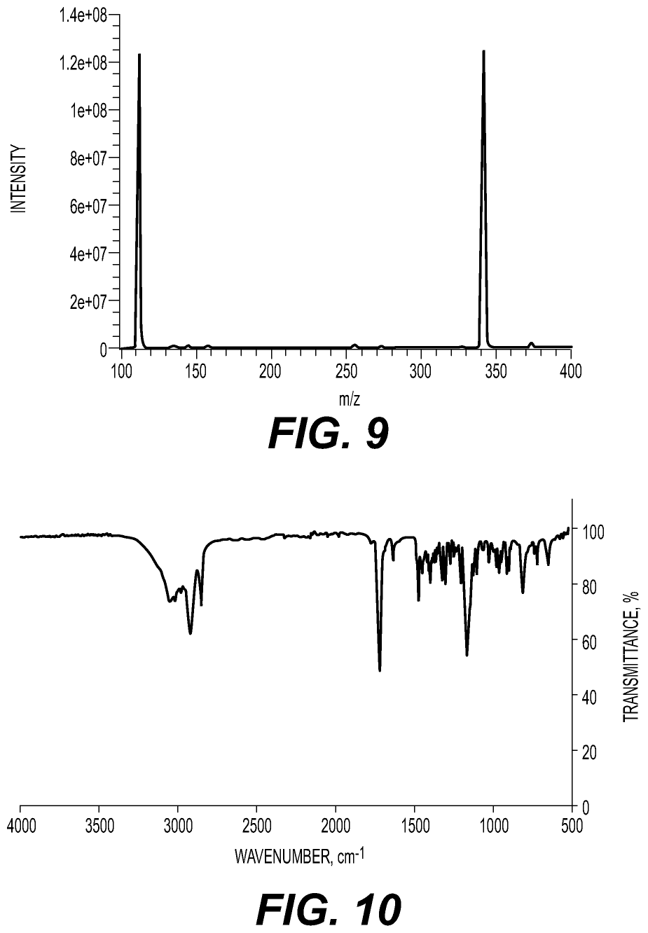

FIG. 9 shows the mass spectra of AMadh2 in the positive ionization mode (MS EI⁺). High abundance fragments, their mass-to-charge (m/z) ratio, and corresponding structures observed were: MSEI⁺ m/z 113.0 (C₆H₉O₂., m/z=113.1), 114.2, 342.4, 343.4, 344.4 (C₁₉H₃₆NO₄⁺, m/z=342.3, 343.3, 344.3).

FIG. 10 shows the FTIR-ATR spectra of AMadh2. Peak positions (νₘₐₓ) of the higher intensity peaks observed are: FTIR-ATR νₘₐₓ 3020.22, 2919.64, 2853.68, 1720.62, 1632.90, 1472.53, 1450.86, 1396.20, 1321.88, 1298.54, 1205.04, 1160.55, 1102.09, 1029.49, 981.36, 961.54, 911.05, 811.23, 718.91, 649.71 cm⁻¹.

Collectively, FTIR, NMR and MS analysis of synthesized AMadh2 compound confirmed that its structure is as designed.

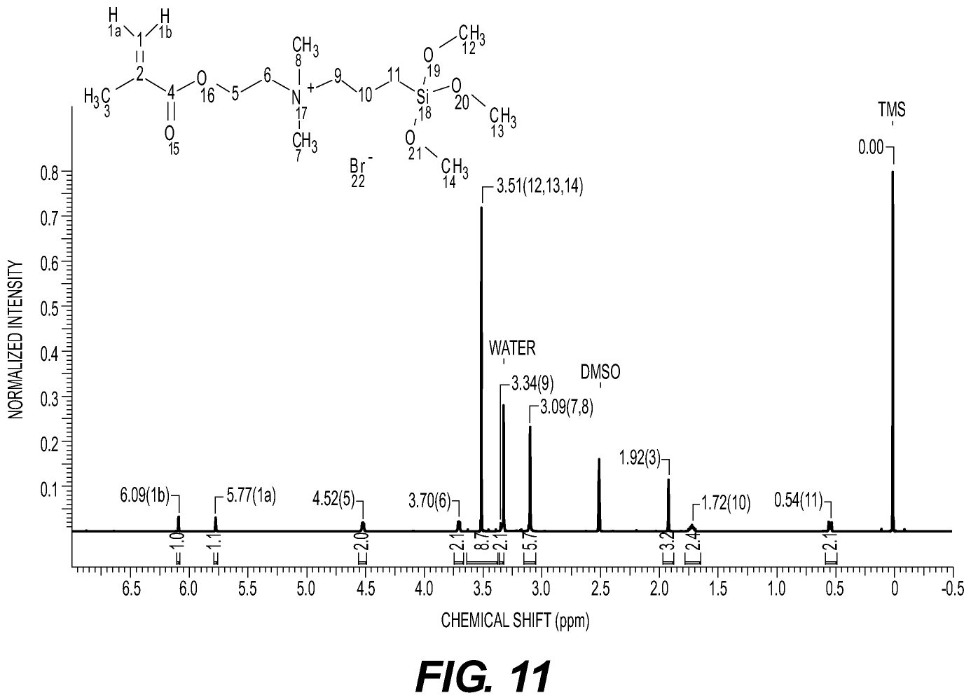

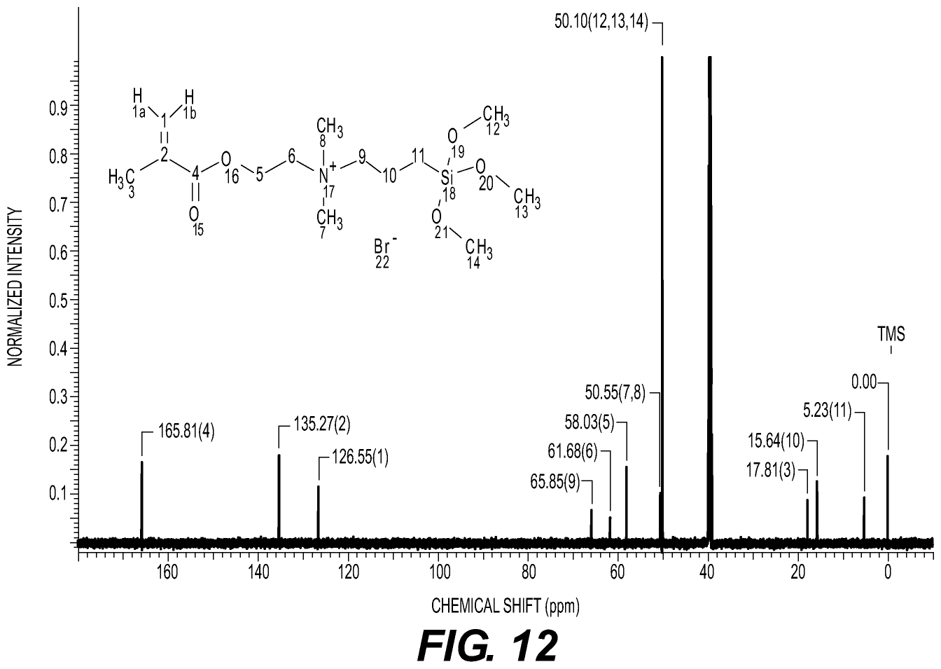

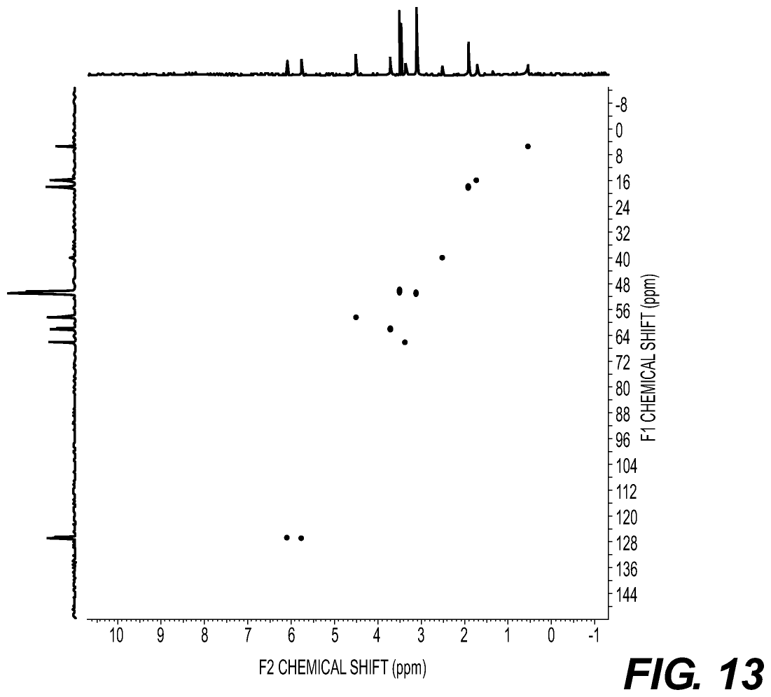

AMsil1 (N-(2-(methacryloyloxy)ethyl)-N,N-dimethyl-3-(trimethoxysilyl)propan-1-aminium iodide). An AMsil1 compound was synthesized as described in the Reaction 3 schematic. FIG. 11 shows its proton (1H) NMR spectra, structure and proton (δH) chemical shifts, assignments and integration values. FIG. 12 shows its carbon (13C) NMR spectra, structure and carbon (δC) chemical shifts and assignments. FIG. 13 shows its HSQC spectra (1H chemical shifts are potted on X-axis(F2) and 13C chemical shifts on Y-axis(F1)). These results confirm successful quaternization of the tertiary amine reactant. Complete NMR structural assignment of AMsil1 compound is given in Table 4.

TABLE 4
NMR Spectroscopic Data for AMsil1.

| position | δC, ppm | δH, ppm |
|---|---|---|
| 1 | 126.6 | 5.77; 6.09 |
| 2 | 135.3 | |
| 3 | 17.8 | 1.92 |
| 4 | 165.8 | |
| 5 | 58.0 | 4.52 |
| 6 | 61.7 | 3.70 |
| 7, 8 | 50.6 | 3.09 |
| 9 | 65.9 | 3.34 |
| 10 | 15.6 | 1.72 |
| 11 | 5.2 | 0.54 |
| 12, 13, 14 | 50.1 | 3.51 |

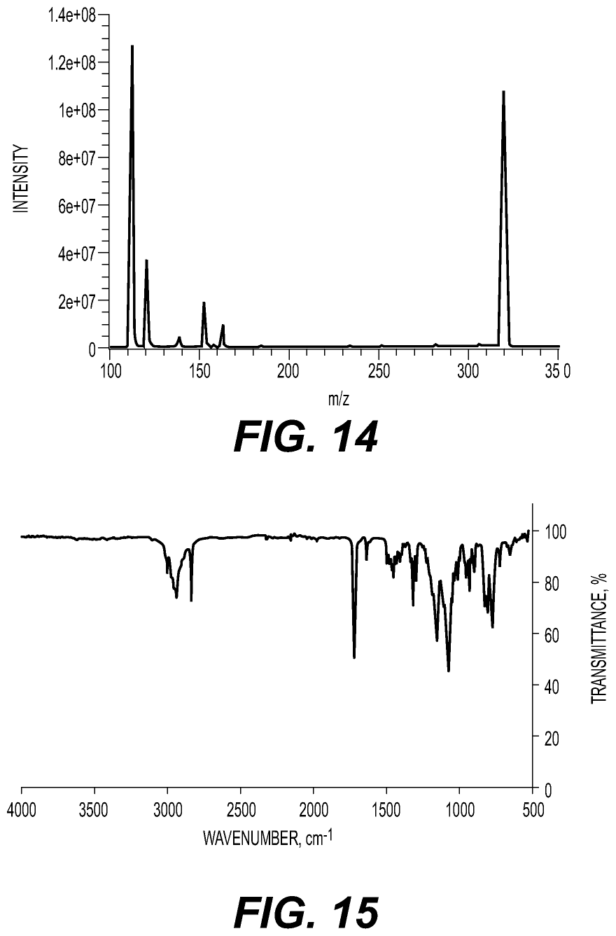

FIG. 14 shows the mass spectra of AMsil1 in the positive ionization mode (MS EI⁺). High abundance fragments, their mass-to-charge (m/z) ratio, and corresponding structures observed were: MSEI⁺ m/z 113.0 (C₆H₉O₂., m/z=113.1), 121.0 (C₃H₉O₃Si., m/z=121.0), 153.1, 163.2 (C₆H₁₅O₃Si., m/z=163.1), 320.3, 321.3, 322.2 (C₁₄H₃₀NO₅Si⁺, m/z=320.2, 321.2, 322.2).

FIG. 15 shows the FTIR-ATR spectra of AMsil1. Peak positions (νₘₐₓ) of the higher intensity peaks observed are: FTIR-ATR νₘₐₓ 3006.36, 2942.53, 2839.78, 1721.33, 1640.85, 1498.52, 1452.65, 1408.73, 1317.51, 1296.44, 1157.04, 1075.87, 1014.52, 955.81, 930.77, 902.10, 827.52, 808.42, 775.28, 727.63, 654.36 cm⁻¹.

Collectively, FTIR, NMR and MS analysis of synthesized AMsil1 compound confirmed that its structure is as designed.

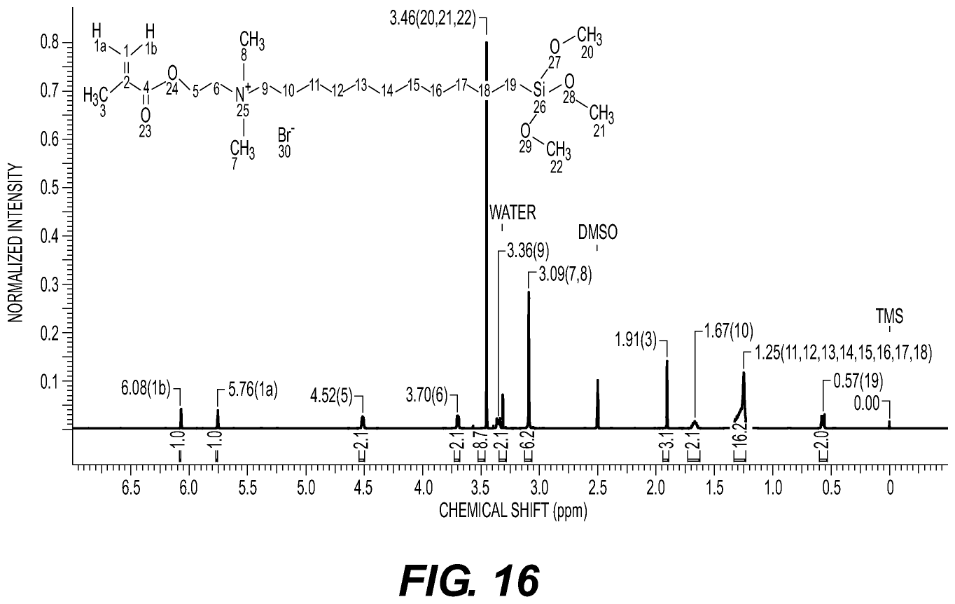

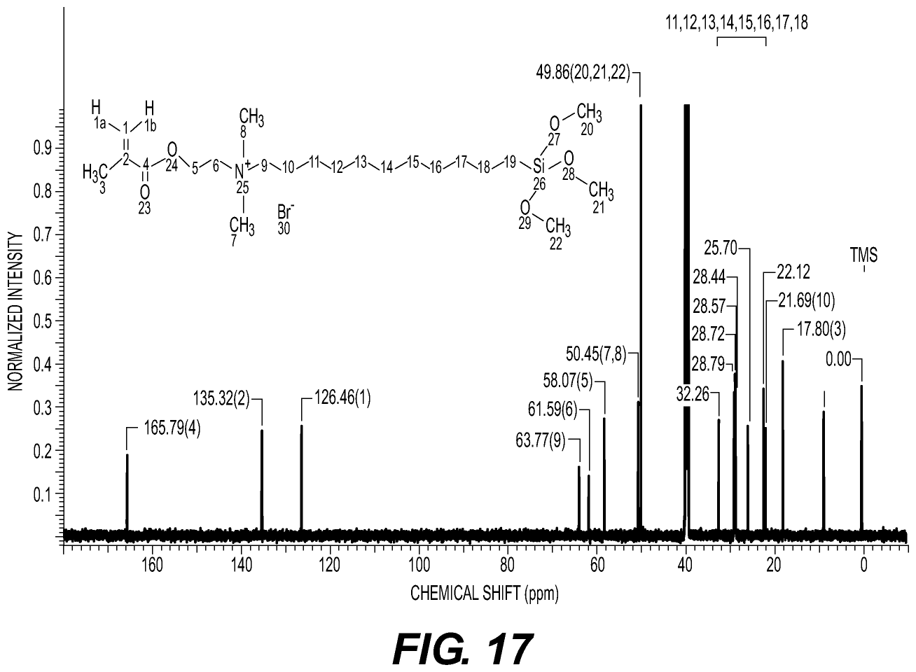

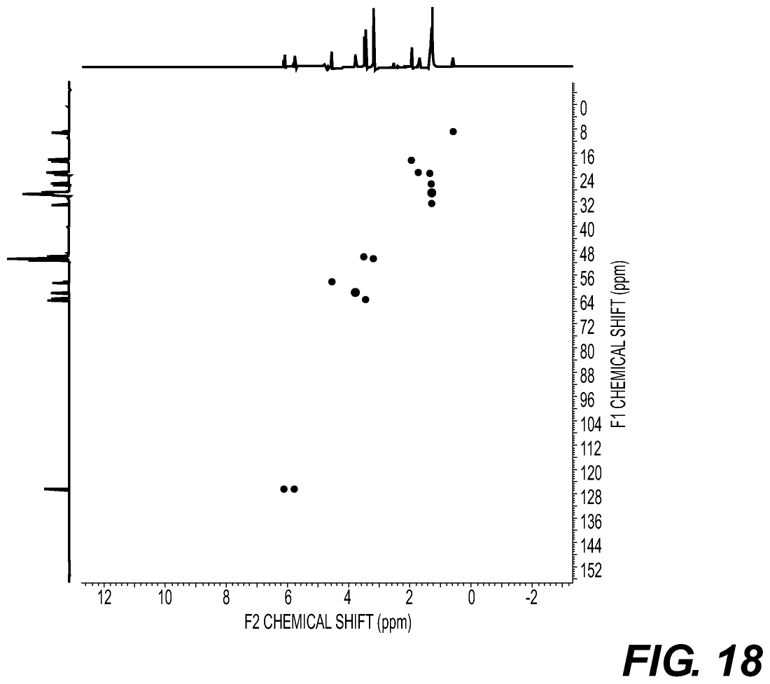

AMsil2 (N-(2-(methacryloyloxy)ethyl)-N,N-dimethyl-11-(trimethoxysilyl)undecan-1-aminium bromide). An AMsil2 compound was synthesized as described in the Reaction 4 schematic. FIG. 16 shows its proton (1H) NMR spectra, structure and proton (δH) chemical shifts, assignments and integration values. FIG. 17 shows its carbon (13C) NMR spectra, structure and carbon (δC) chemical shifts and assignments. FIG. 18 shows its HSQC spectra (1H chemical shifts are potted on X-axis(F2) and 13C chemical shifts on Y-axis(F1)). These results confirm successful quaternization of the tertiary amine reactant. Complete NMR structural assignment of AMsil2 compound is given in Table 5.

TABLE 5
NMR Spectroscopic Data for AMsil2.

| position | δC, ppm | δH, ppm |
|---|---|---|
| 1 | 126.5 | 5.76; 6.08 |
| 2 | 135.3 | |
| 3 | 17.8 | 1.91 |
| 4 | 165.8 | |
| 5 | 58.1 | 4.52 |
| 6 | 61.6 | 3.70 |
| 7, 8 | 50.4 | 3.09 |
| 9 | 63.8 | 3.36 |
| 10 | 21.7 | 1.67 |
| 11-18 | 22.1, 25.7, 28.4, 28.6, 28.7, 28.8, | |
| | 28.9, 32.3 | 1.25 |
| 19 | 8.6 | 0.57 |
| 20, 21, 22 | 49.9 | 3.46 |

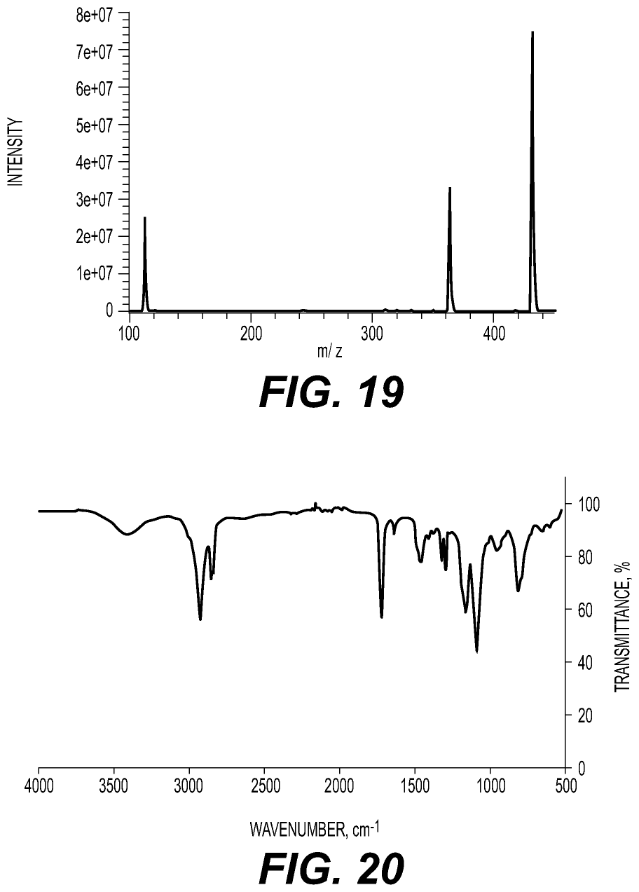

FIG. 19 shows the mass spectra of AMsil2 in the positive ionization mode (MS EI⁺). High abundance fragments, their mass-to-charge (m/z) ratio, and corresponding structures observed were: MSEI⁺ m/z 113.0 (C₆H₉O₂., m/z=113.1), 114.2, 364.3, 365.4, 366.4 (C₁₈H₄₂NO₄Si.⁺, m/z=364.3, 365.3, 366.3), 432.4, 433.4, 434.4 (C₂₂H₄₆NO₅Si⁺, m/z=432.3, 433.3, 434.3).

FIG. 20 shows the FTIR-ATR spectra of AMsil1. Peak positions (νₘₐₓ) of the higher intensity peaks observed are: FTIR-ATR νₘₐₓ 3401.44, 2924.91, 2853.72, 1720.62, 1637.82, 1465.22, 1318.37, 1295.37, 1159.98, 1090.00, 953.85, 811.57, 655.18, 600.60 cm⁻¹.

Collectively, FTIR, NMR and MS analysis of synthesized AMsil2 compound confirmed that its structure is as designed.

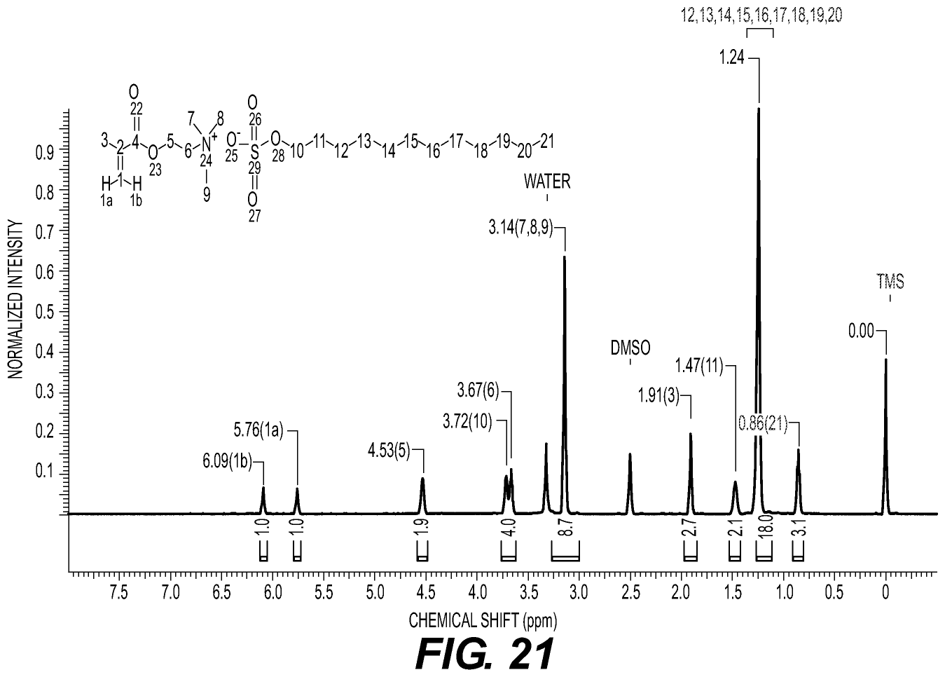

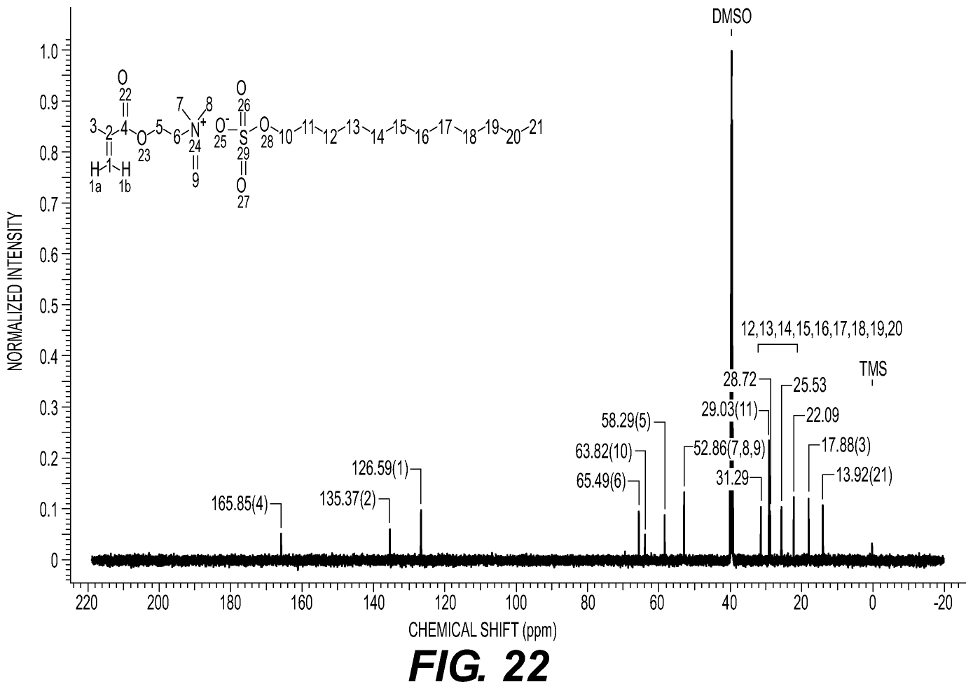

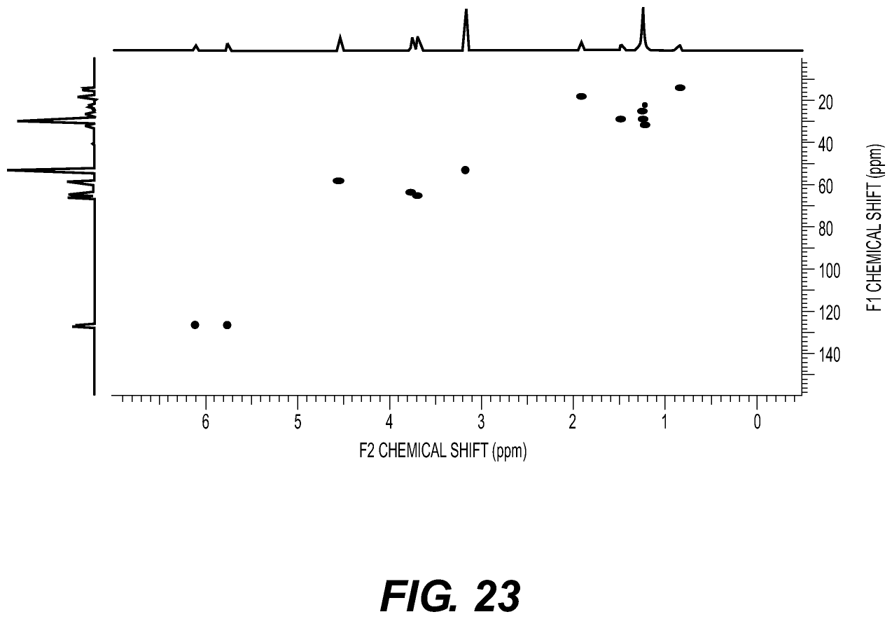

AMmis1 (2-(methacryloyloxy)-N,N,N-trimethylethan-1-aminium dodecyl sulfate). An AMmis1 compound was synthesized as described in the Reaction 5 schematic. FIG. 21 shows its proton (1H) NMR spectra, structure and proton (δH) chemical shifts, assignments and integration values. FIG. 22 shows its carbon (13C) NMR spectra, structure and carbon (δC) chemical shifts and assignments. FIG. 23 shows its HSQC spectra (1H chemical shifts are potted on X-axis(F2) and 13C chemical shifts on Y-axis(F1)). These results confirm successful substitution of chloride anion of the quaternary amine reactant with dodecyl sulfate anion. Complete NMR structural assignment of AMmis1 compound is given in Table 6.

TABLE 6
NMR Spectroscopic Data for AMmis1.

| position | δC, ppm | δH, ppm |
|---|---|---|
| 1 | 126.6 | 6.09, 5.76 |
| 2 | 135.4 | |
| 3 | 17.9 | 1.91 |
| 4 | 165.9 | |
| 5 | 58.3 | 4.53 |
| 6 | 65.5 | 3.66 |
| 7, 8, 9 | 52.9 | 3.14 |
| 10 | 63.8 | 3.71 |
| 11 | 29.0 | 1.47 |
| 12-20 | 22.1, 25.5, 28.7, 31.3 | 1.24 |
| 21 | 13.9 | 0.86 |

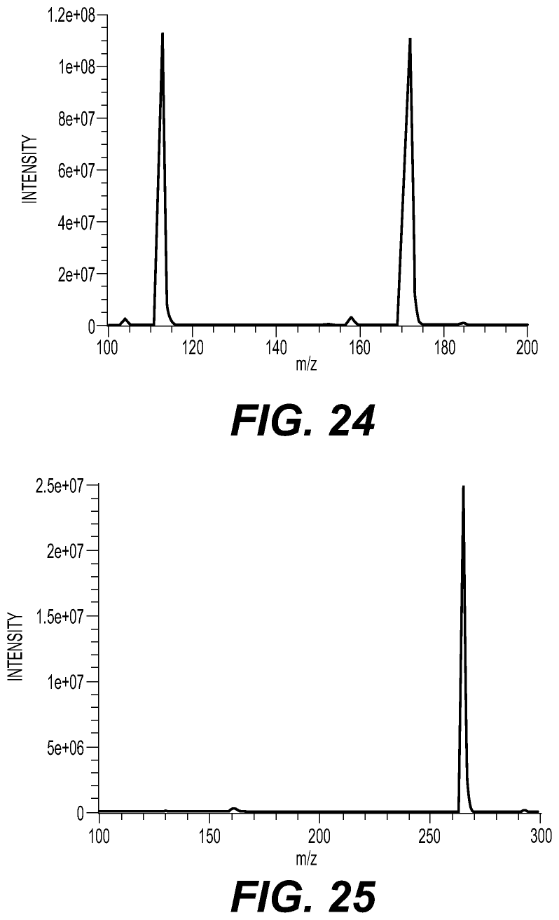

FIG. 24 shows the mass spectra of AMmis1 in the positive ionization mode (MS EI⁺). FIG. 25 shows the mass spectra of AMmis1 in the negative ionization mode (MS EI⁻). High abundance fragments, their mass-to-charge (m/z) ratio, and corresponding structures observed were: MSEI⁺ m/z 113.0 (C₆H₉O₂., m/z=113.1), 158.1, 172.2, 173.2, (C₉H₁₈NO₂⁺, m/z=172.1, 173.1), and MSEI⁻ m/z 265.3, 266.3, 267.3 (C₁₂H₂₅O₄S⁻, m/z=265.1, 266.2, 267.1).

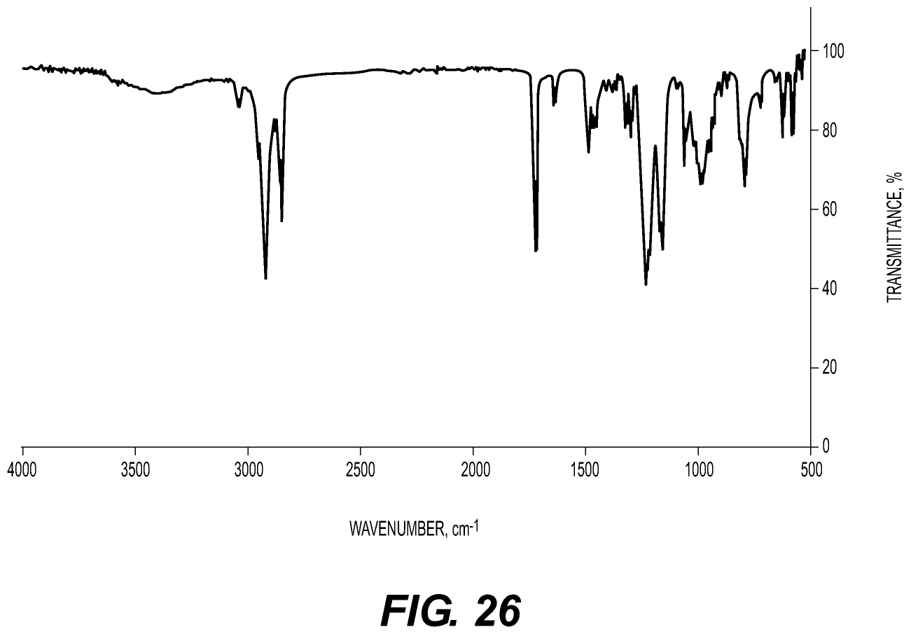

FIG. 26 shows the FTIR-ATR spectra of AMmis1. Peak positions (νₘₐₓ) of the higher intensity peaks observed are: FTIR-ATR νₘₐₓ 573.8, 581.1, 601.3, 622.5, 656.0, 721.8, 790.8, 870.2, 895.4, 934.3, 948.3, 987.1, 1018.6, 1059.4, 1096.1, 1155.6, 1166.7, 1214.9, 1229.7, 1295.8, 1321.9, 1346.9, 1363.3, 1378.5, 1405.9, 1455.8, 1467.9, 1485.8, 1640.5, 1720.7, 2851.4, 2920.1, 2953.9, 3039.7, 3400.0, 3580.9 cm⁻¹.

Collectively, FTIR, NMR and MS analysis of synthesized AMmis1 compound confirmed that its structure is as designed.

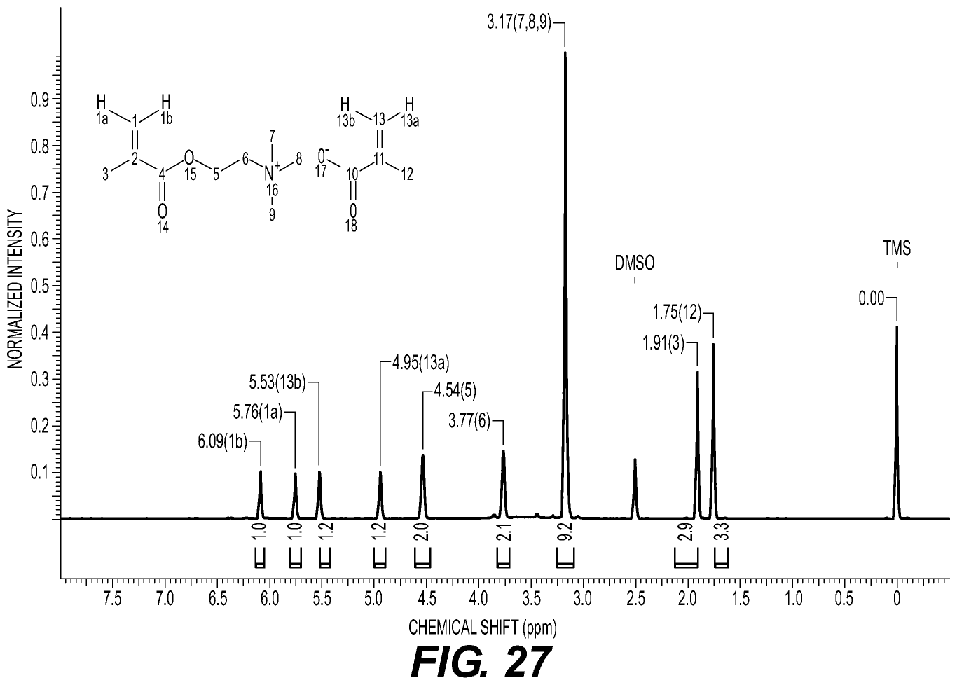

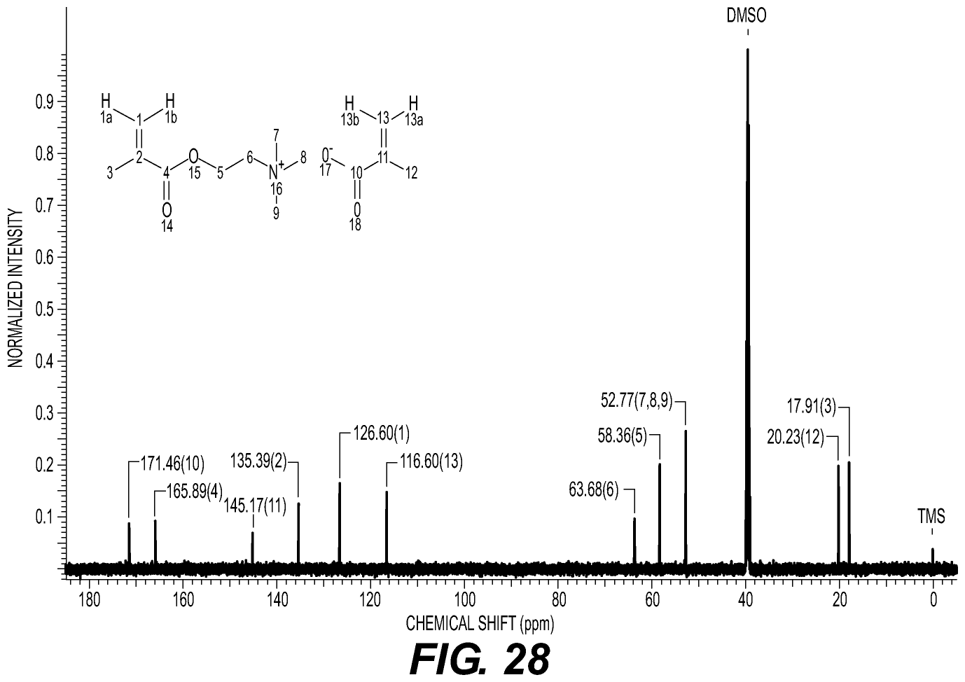

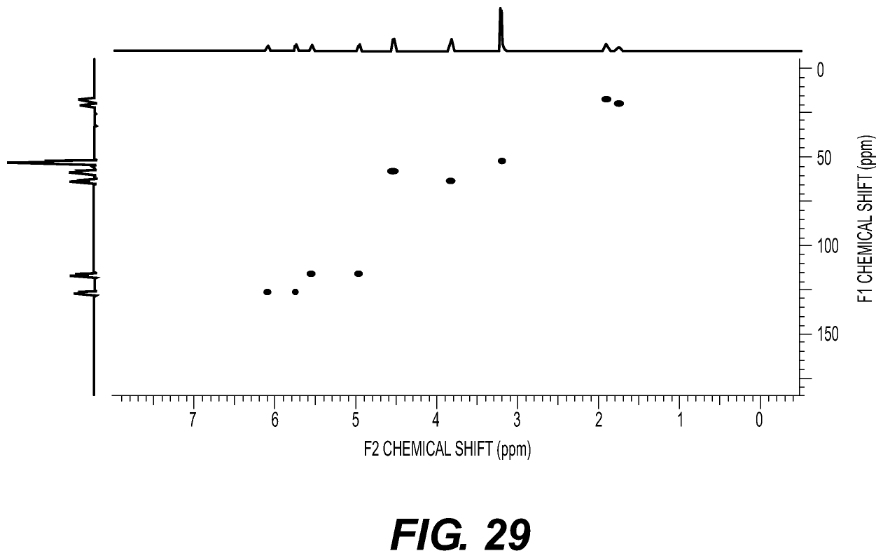

AMmis2 (2-(methacryloyloxy)-N,N,N-trimethylethan-1-aminium methacrylate). An AMmis2 compound was synthesized as described in the Reaction 6 schematic. FIG. 27 shows its proton (1H) NMR spectra, structure and proton (δH) chemical shifts, assignments and integration values. FIG. 28 shows its carbon (13C) NMR spectra, structure and carbon (δC) chemical shifts and assignments. FIG. 29 shows its HSQC spectra (1H chemical shifts are potted on X-axis(F2) and 13C chemical shifts on Y-axis(F1)). These results confirm successful substitution of chloride anion of the quaternary amine reactant with methacrylate anion. Complete NMR structural assignment of AMmis2 is given in Table 7.

TABLE 7
NMR Spectroscopic Data for AMmis2

| position | δC, ppm | δH, ppm |
|---|---|---|
| 1 | 126.6 | 6.09, 5.76 |
| 2 | 135.4 | |
| 3 | 17.9 | 1.91 |
| 4 | 165.9 | |
| 5 | 58.4 | 4.54 |
| 6 | 63.7 | 3.77 |
| 7, 8, 9 | 52.8 | 3.17 |
| 10 | 171.5 | |
| 11 | 145.2 | |
| 12 | 20.2 | 1.75 |
| 13 | 116.6 | 5.53, 4.95 |

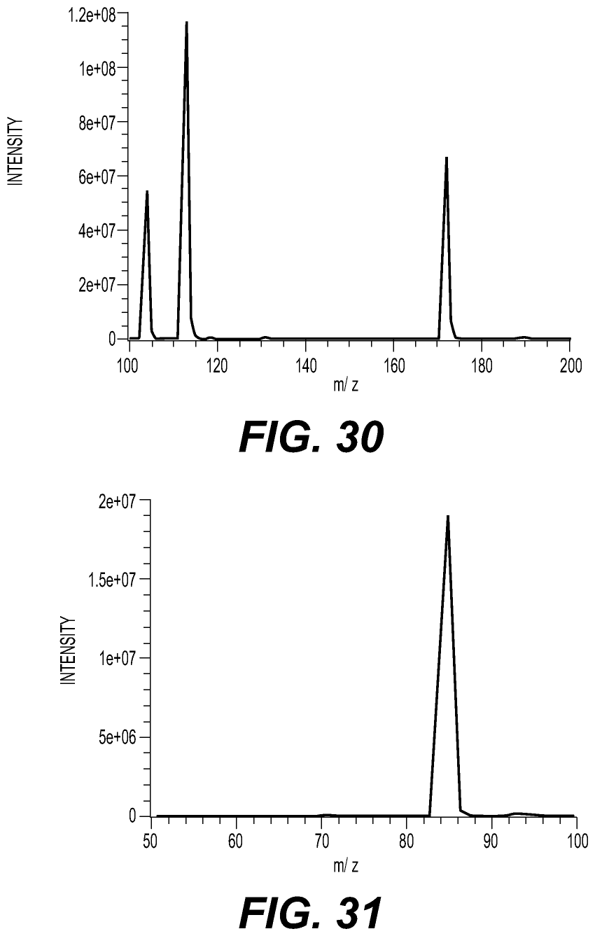

FIG. 30 shows the mass spectra of AMmis2 in the positive ionization mode (MS EI⁺). FIG. 31 shows the mass spectra of AMmis2 in the negative ionization mode (MS EI⁻). High abundance fragments, their mass-to-charge (m/z) ratio, and corresponding structures observed were: MSEI⁺ m/z 104.0, 113.0 (C₆H₉O₂., m/z=113.1), 172.2, 173.2, (C₉H₁₈NO₂⁺, m/z=172.1, 173.1), and MSEI⁻ m/z 85.0 (C₄H₅O₂⁻, m/z=85.0, 86.0).

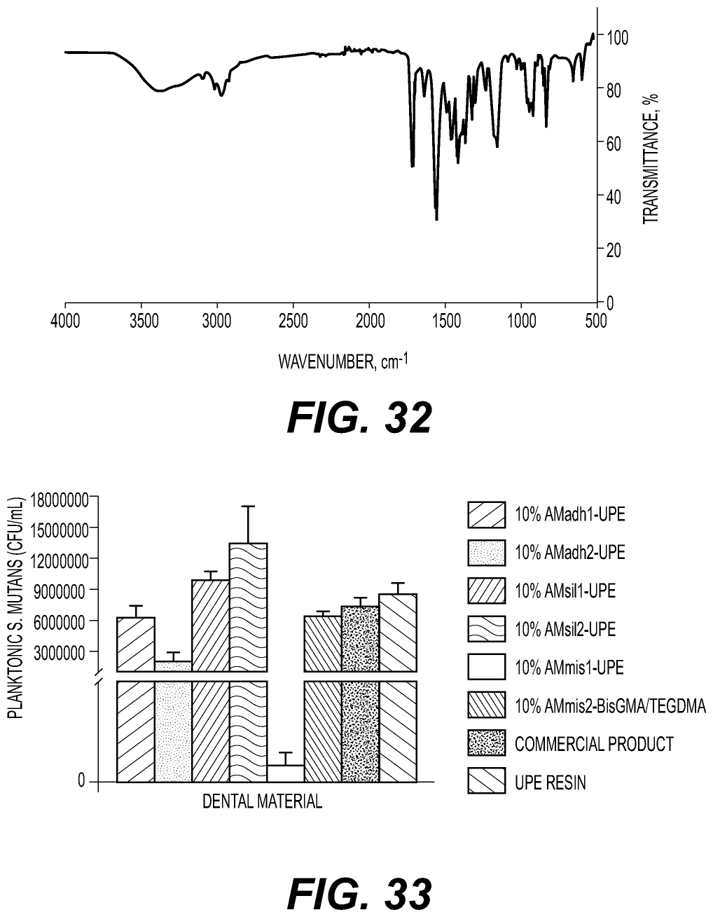

FIG. 32 shows the FTIR-ATR spectra of AMmis2. Peak positions (νₘₐₓ) of the higher intensity peaks observed are: FTIR-ATR νₘₐₓ 601.1, 659.0, 813.7, 835.3, 856.0, 895.7, 921.0, 946.4, 961.4, 986.8, 1002.2, 1030.1, 1087.7, 1157.6, 1233.2, 1295.9, 1322.3, 1367.7, 1387.0, 1403.4, 1417.6, 1457.6, 1491.4, 1555.6, 1638.5, 1715.5, 2929.6, 2969.8, 3019.0, 3090.0, 3375.6 cm⁻¹.

Collectively, FTIR, NMR and MS analysis of synthesized AMmis2 compound confirmed that its structure is as designed.

### Experimental Series 2: Physicochemical and Mechanical Properties of Copolymer Resins Containing AM Monomers

### Materials and Methods:

Experimental resin (UPE) was formulated from the commercially available monomers urethane dimethacrylate (UDMA, 50.91% w/w), polyethylene glycol extended urethane dimethacrylate (PEG-U, 18.18% w/w), and ethyl 2-(hydroxymethyl)acrylate (EHMA, 30.91% w/w). A conventional visible light initiator system comprised of camphorquinone (0.2% w/w) and ethyl-4-N,N-dimethylamino benzoate (4EDMAB, 0.8% w/w) was utilized. Quaternary ammonium monomers were blended into UPE resin to yield (AMadh1, AMadh2, AMsil1, AMsil2, AMmis1)-UPE resins with 10 or 20 mass % of AM component. Copolymer specimens (beams) were prepared by depositing UPE (UPE resin control) and AM-UPE resins into glass molds (2 mm×2 mm×25 mm) and photopolymerizing them with a dental curing light (Triad 2000, Dentsply, Charlotte, N.C., USA) for 2 minutes from two sides. Copolymer specimens (pellets) were prepared by depositing UPE and AM-UPE resins into a stainless-steel mold (1 mm×16 mm), sandwiched between two sheets of Mylar film and two glass slides and photopolymerized in the same manner as the beam samples. Degree of vinyl conversion (DVC) of UPE and AM-UPE resins was determined utilizing beam samples by collecting the near infra-red (NIR) spectra (Nexus 670, ThermoFisher, Madison, Wis., USA) before and 24 h after polymerization and calculating the reduction in absorption band at 6165 cm⁻¹ corresponding to the vinyl (C═C—H) overtone signal. By maintaining a constant specimen thickness, a need for an invariant internal standard was eliminated. The DVC was calculated as:

$$DVC ( \% ) = 100 \times [ 1 - \frac{Area ( polymer )}{Area ( monomer )} ]$$

where Area(polymer) and Area(monomer) correspond to the areas under 6165 cm⁻¹ peaks after and before the polymerization, respectively. After DVC analysis, flexural strength (FS) and elastic modulus (E) of beam specimen were determined employing the Universal Testing Machine (Instron 5500R, Instron Corp., Canton, Mass., USA). The load was applied (at cross-head speed of 1 mm/min) to the center of a specimen positioned on a test device with supports 20 mm apart. The FS and E of the specimens (three replicates/experimental group) was calculated as instructed in the ISO4049:2009 document. Changes in hydrophilicity/hydrophobicity of UPE resins due to the introduction of AM monomers were assessed on pellet specimen by sessile drop contact angle (CA) measurements (drop shape analyzer DSA100, Krüss GmbH, Hamburg, Germany). Following the deposition of 4 μL water droplets on the resin pellets, the pellets were imaged after 1 min resting time with a camera at the points of intersection (three-phase contact points) between the drop contour and the projection of the surface (baseline). The CA water values were calculated employing the Krüss Advance software. At least four repetitive measurements were performed in each group. Analysis of variance (ANOVA) and Newman-Keuls multiple paired comparisons (at 95% confidence level) were used to analyze the experimental data as a function of resin composition and establish a statistical significance of differences between the experimental groups (WINKS 7.0.9, TexaSoft, Cedar Hills, Tex., USA).

### Results:

Table 8 lists physicochemical and mechanical properties of UPE copolymer resins containing disclosed AM monomers. Values marked with *, **, *** or **** indicate statistically significant differences within each column between different resin formulations (Newman-Keuls multiple comparisons test, p<0.05).

TABLE 8
Physicochemical and Mechanical Properties of UPE Resins Containing Disclosed AM Monomers.

| Resin | DVC, % | FS, MPa | E, MPA | CA, ° |
|---|---|---|---|---|
| UPE (100% w/w) | 88.1 ± 0.9 *** | 94.8 ± 5.0 | 2292.5 ± 104.0 **** | 60.8 ± 5.1 ****/*** |
| (control) | | | | |
| UPE (90% w/w) - | 90.1 ± 0.8 *** | 76.6 ± 4.6 ***/** | 2175.6 ± 203.4**** | 56.5 ± 2.6 ****/***/** |
| AMadh1 (10% w/w) | | | | |
| UPE (80% w/w) - | 91.1 ± 0.5 *** | 67.9 ± 3.3 *** | 2274.2 ± 176.4 **** | 64.8 ± 2.7 **** |
| AMadh1 (20% w/w) | | | | |
| UPE (90% w/w) - | 88.5 ± 2.5 *** | 83.8 ± 4.9 ** | 2724.3 ± 99.7 ** | 42.4 ± 8.5 **/* |
| AMadh2 (10% w/w) | | | | |
| UPE (80% w/w) - | 85.7 ± 2.6 *** | 70.5 ± 2.7 *** | 3003.8 ± 63.4 * | 64.6 ± 6.4 **** |
| AMadh2 (20% w/w) | | | | |
| UPE (90% w/w) - | 60.7 ± 4.0 | 84.6 ± 4.7 ** | 2000.7 ± 194.7 ****/*** | 46.9 ± 5.9 ***/**/* |
| AMsil1 (10% w/w) | | | | |
| UPE (80% w/w) - | 70.9 ± 11.0** | 42.3 ± 3.3 * | 981.6 ± 70.5 | 51.8 ± 11.2 ****/***/**/* |
| AMsil1 (20% w/w) | | | | |
| UPE (90% w/w) - | 86.7 ± 2.0 *** | 82.6 ± 2.5 ** | 2016.5 ± 79.5 ****/*** | 37.4 ± 9.2 * |
| AMsil2 (10% w/w) | | | | |
| UPE (80% w/w) - | 75.2 ± 6.9 ** | 70.8 ± 2.8 *** | 1715.7 ± 74.0 *** | 53.3 ± 6.7 ****/***/** |
| AMsil2 (20% w/w) | | | | |
| UPE (90% w/w) - | 86.4 ± 0.8 *** | 72.3 ± 5.4 *** | 1832.2 ± 171.2 *** | 37.1 ± 11.4**/* |
| AMmis1 (10% w/w) | | | | 64.8 ± 2.4 **** |

### Degree of Vinyl Conversion:

### AMadh Series Monomers:

Incorporation of an AMadh1 monomer at 10 and 20 mass percent into UPE resin increased the degree of vinyl conversion of produced copolymers by 2% and 3%, respectively. However, these results were not statistically different relative to a UPE resin control (p>0.05). Incorporation of AMadh2 monomer at 10 and 20 mass percent into UPE maintained the degree of vinyl conversion similar to that of the UPE resin control (p>0.05).

### AMsil Series Monomers:

Incorporation of AMsil1 monomer at 10 and 20 mass percent into UPE resin reduced the degree of vinyl conversion of produced copolymers by approximately 27% and 17%, respectively. While these results were statistically different from each other, the increased amount (20% by mass) of AMsil1 monomer did not cause dose dependent reduction in DVC. Incorporation of AMsil2 at 10 mass percent did not change the copolymer DVC relative to UPE resin control. However, increasing amount of AMsil2 monomer to 20 mass percent reduced the DVC by 12.9% compared to UPE resin control (p<0.05).

### AMmis1 Monomer:

Incorporation of AMmis1 monomer at 10 mass percent maintained the DVC of copolymer at the same level as the UPE resin control (p>0.05).

### Flexural Strength (FS) and Elastic Modulus (E):

### AMadh Series Monomers:

Incorporation of AMadh1 monomer at 10 and 20 mass percent reduced FS of the copolymer by 20% and 28% relative to the UPE resin control, although no statistically significant difference in FS was observed between these formulations. The elastic modulus (E) of these copolymers was not affected by AMadh1 monomer. Incorporation of AMadh2 monomer at 10 and 20 mass percent also reduced the FS of the copolymers, however, increased the elastic modulus (E) of copolymers by 432 and 711 MPa, respectively.

### AMsil Series Monomers:

The FS and E of UPE-AMsil copolymers were, generally, diminished compared to the UPE resin control. The extent of reduction in FS and E varied with the type and the concentration of AMsil. FS and E data, expectedly, showed similar trends (Table 8). In all but a UPE(90% w/w)-AMsil1(10% w/w) formulation, the FS and the E values were significantly (p<0.05) lower than the UPE resin control. The reductions ranged from moderate (11-13% for 10 mass % AMsil formulations) to substantial (56-57% for 20 mass % AMsil formulations).

### AMmis1 Monomer:

The FS and E of UPE(90% w/w)-AMmis1(10% w/w) copolymer was reduced by 24% and 460 MPa relative to UPE resin control.

### Contact Angle:

### AMadh Series Monomers:

Incorporation of AMadh1 monomer at 10 and 20 mass percent and AMadh2 monomer at 20 mass percent into UPE resin did not alter the hydrophobic-hydrophilic properties of the copolymers (p>0.05). AMadh2 monomer at 10 mass percent reduced the water CA of copolymer by 18°, making it more hydrophilic.

### AMsil Series Monomers:

Copolymers comprised of UPE resin with added AMsil monomers, generally exhibited lower contact angles (CA), suggesting changes in their hydrophilic/hydrophobic balance toward more hydrophilic surfaces. At 10 mass % AMsil monomers in the resin, CAs of both AMsil1 and AMsil2 containing copolymers (46.9±5.9° and 37.4±9.2°, respectively) were significantly lower (23% and 38% reduction, respectively; p≤0.05) than the CA of the UPE resin control 60.8±5.1°. The apparent increase in the CA in going from 10 to 20% AMsil in the resin was significant only for AMsil2 (p≤0.05). The overall order of the decreased relative hydrophilicity (evidenced by the increasing CA values) of the examined UPE-based copolymers was as follows: (UPE-AMsil2 (10% w/w) UPE-AMsil1 (10% w/w)>UPE-AMsil2 (20% w/w)=UPE-AMsil1 (10% w/w))>UPE resin control.

### AMmis1 Monomer:

A UPE copolymer containing AMmis1 monomer at 10 mass percent exhibited amphiphilic surface properties with one side of the copolymer pellet becoming more hydrophilic (CA=37.1±11.4°) and the other retaining hydrophobic-hydrophilic balance (CA=64.8±2.4°) similar to the UPE resin control.

The range of DVC values attained in UPE-AM copolymers (60.7-91.1%) was dependent on both the monomer type and its quantity in the resin, and were higher or equal to the DVC reported for 2,2-bis[p-(2-hydroxy-3-methacryloxypropoxy)phenyl]propane (Bis-GMA)-based resins/composites with incorporated QA ionic dimethacrylate, currently utilized in dental practice (67.9-70.7%) [Antonucci, J. M.; Zeiger, D. N.; Tang, K.; Lin-Gibson, S.; Fowler, B. O.; Lin, N. J. Synthesis and characterization of dimethacrylates containing quaternary ammonium functionalities for dental applications. Dent. Mater. 2012, 28, 219-228, 2011]. AMadh containing copolymers reached significantly higher DVC values (85.7-91.1%) compared to their AMsil containing counterparts (60.7-86.7%). The high DVCs attained in UPE-AM copolymers suggest limited mobility of cross-linked polymer matrix thus reducing the likelihood of unreacted monomers leaching out to a minimal value.

The results of flexural strength and elastic modulus tests indicated a reduction of the copolymers' mechanical properties in going from the UPE resin control to UPE-AM formulations. This overall reduction in mechanical properties does not disqualify the UPE-AM formulations from use in dental applications. In accordance with the International Standard (ISO4049:2009), which specifies requirements for dental polymer-based filling and restorative materials and intended for use primarily for the direct or indirect restoration of cavities in the teeth, all disclosed UPE-AM formulations meet the flexural strength requirement of 50 MPa necessary for type 2 (all polymer-based restorative materials not intended for occlusal restorations), class 2 (materials whose setting is effected by the application of energy from an external source, such as blue light or heat) formulations. To further compensate for the reduction in the mechanical properties, incorporation of the reinforcing fillers commonly utilized in dentistry may be considered (for example silica oxide, titanium oxide, etc.).

Introduction of AMadh1 (at 10 and 20 mass percent), AMadh2 (at 20 mass percent), AMsil1 (at 20 mass percent) and AMsil2 (at 20 mass percent) monomers maintained the hydrophobic-hydrophilic balance of the UPE resin control. Hydrophobicity of these copolymers is expected to minimize their interaction with water molecules, therefore minimizing water sorption in oral environment. Introduction of AMadh2 (at 10 mass percent), AMsil1 (at 10 mass percent) and AMsil2 (at 10 mass percent) into UPE resin, a shift towards the lower CA values, consistent with the moderate increase in the overall hydrophilicity, was seen. The hydrophilic nature of these copolymers makes them good candidates for the incorporation of filler materials, especially remineralizing fillers (for example, amorphous calcium phosphate). Their enhanced wettability should ease diffusion of water into the composite and result in the subsequent release from the composite of calcium and phosphate ions needed for tooth demineralization prevention and/or remineralization at the restoration site. Introduction of AMmis1 monomer (at 10 mass percent) resulted in formation of amphiphilic copolymer with one hydrophilic and one hydrophobic surface. This phase re-arrangement appears to occur during a photopolymerization step and is yet not well understood. However, such amphiphilic properties of the copolymer are desirable when the copolymer is intended to be in simultaneous contact with a hydrophilic surface/material (for example tooth enamel or dentin) and hydrophobic surface/material (for example hydrophobic dental composites). Overall, the detected range of CAs in UPE-AM resins (37.4-53.3°) correlates very well with the range of CAs typical for the commercial resin composites (37.4-64.8°) [da Silva, E. M.; Almeida, G. S.; Poskus, L. T.; Guimaraes, J. G. Relationship between the degree of conversion, solubility and salivary sorption of a hybrid and a nanofilled resin composite. J. Appl. Oral Sci. 2008, 16, 161-166].

### Experimental Series 3: Antimicrobial Effect of Copolymer Resins Containing AM Monomers Against Planktonic Forms of Streptococcus mutans

### Methods:

Testing of Streptococcus mutans (S. mutans, UA-159 (ATCC® 700610)) planktonic forms was according to described methods¹ (references noted subsequently herein are listed later in a References section). Briefly, S. mutans stock cultures were propagated using Todd Hewitt broth (THB) (Becton Dickinson and Co., Sparks, Md., USA). Cultures with an optical density of 1.2-1.3 at 600 nm (Unico® 1200 Spectrophotometer, United Products & Instruments, Inc., Dayton, N.J., USA) were diluted in Dulbecco's phosphate-buffered saline to achieve ˜10¹⁰ colony-forming units (CFUs)/mL. Copolymer disks were seeded with 3 μL of the S. mutans suspension (˜3×10⁷ CFU/disk). To maximize the contact between the copolymer and bacteria, a second disk was placed atop (e.g., sandwich). This assembly was incubated at 37° C. in a 5% CO₂ environment for 2 h. Afterwards, the samples were placed in 1 mL of THB, vortexed, and utilized to prepare a 10-fold dilution series. Of the resulting suspensions, 100 μL were distributed onto the surface of THB agar plates. After ˜20 h of incubation, the CFUs were enumerated using an IncuCount Colony Counter (Revolutionary Science, Shafer, Minn., USA). UPE resin disks were used as a negative control group. HemCon® Dental Dressing (HemCon Medical Technologies, Inc., Portland, Oreg., USA) and Clearfil SE Protect adhesive system (Kuraray America, New York, N.Y., USA) was used as a positive kill control. These products were chosen as the proposed mechanism of action (e.g., electrostatic forces between negatively-charged microbial cell membranes and positively-charged chitosan molecules²) is believed to be similar to that of quaternary ammonium methacrylates. The range considered countable was 30 to 300 CFU per spread plate. As previously reported³, agar plates streaked with neat solutions of some groups yielded less than the lower limit of detection. Such data were reported as less than the limit of quantification. The number of CFU/mL was calculated as: CFU number/(volume plated)×(dilution). Mean+standard error counts were obtained from 5 to 6 copolymer disk sandwiches, tested independently.

### Results:

### AMadh1 and AMadh2:

For 10% mass AMadh1-UPE copolymers, the mean number of S. mutans (CFU/mL) was ˜25% lower than the UPE resin control; however, this was insignificant (FIG. 33). FIG. 33 illustrates planktonic S. mutans forms (CFU/mL) after exposure to 10% mass AMadh1-UPE, AMadh2-UPE, AMsil1-UPE, AMsil2-UPE, AMmis1-UPE, or AMmis2-BisGMA/TEGDMA copolymers. The commercial product (Clearfil SE Protect) was an antimicrobial dental restorative containing methacryloyloxydodecyl pyrimidinium bromide. Negative control group consisted of UPE resin. Bar height indicates the mean value of CFU/ml+standard error. The mean CFU/mL of the UPE resins containing 10% AMadh2 was ≥4.0-fold less (P≤0.003) than the UPE resin control and ≥3.5-fold less (P≤0.01) than the commercial product. The number of CFU/mL observed in the AMadh2-UPE groups was notably lower (P≤0.03) than that observed with AMadh1-UPE.

### AMsil1 and AMsil2:

For planktonic bacterial testing, the number of planktonic S. mutans CFU/mL observed amongst the 10% mass AMsil-UPE groups was not statistically different from one another or the control groups (UPE only and commercial antimicrobial dental restorative containing methacryloyloxydodecyl pyrimidinium bromide (FIG. 33).

### AMmis1 and AMmis2:

Planktonic S. mutans testing indicated that antimicrobial activity of 10% AMmis1-UPE copolymer disks was >450-fold greater (P≤0.0001) than the UPE resin control or the commercial antimicrobial dental restorative containing methacryloyloxydodecyl pyrimidinium bromide (FIG. 33). Conversely, the number of S. mutans CFU/mL observed amongst the AMmis2-BisGMA-TEGDMA group was not statistically different from UPE resin control or the commercial antimicrobial dental restorative containing methacryloyloxydodecyl pyrimidinium bromide).

Conclusion. Compared to the commercial antimicrobial dental restorative, AMadh2-UPE and AMmis1-UPE copolymers were most effective at reducing the number (CFU/mL) of planktonic S. mutans. Antimicrobial functionality of AMadh1-UPE, AMsil1-UPE, AMsil2-UPE, and AMmis2-BisGMA-TEGDMA copolymers against planktonic S. mutans were comparable to the commercial antimicrobial product and the UPE resin control.

Antimicrobial Effect of Copolymer Resins Containing AM Monomers Against Biofilm Forms of Streptococcus mutans:

### Methods:

A bioluminescent Streptococcus mutans (S. mutans) strain JM 10 (derivative of wild type UA159)⁴ was used to assess the AM properties of AMadh series monomers and AMsil series monomers. Methods of real-time bioluminescence assay were as described^{5, 6}. Briefly, overnight cultures were created by placing a single colony of S. mutans into 4 mL THY medium (THB with yeast extract) containing 32 μL of spectinomycin and incubated at 37° C. for 16-18 h. Overnight cultures exhibiting an OD₆₀₀≥0.900 were used for growing biofilms. A 1:100 dilution using the overnight culture and 0.65×THY+1% sucrose was added (1 mL) to each well containing a copolymer disk. The specimens then were incubated at 37° C. for 24 h in a candle jar. Following incubation, the specimens were removed, and the media was aspirated. The specimens were washed with sterile phosphate-buffered saline (3 times at 15 seconds/wash, 150 rpm), and transferred to a sterile, white 24-well plate. 1 mL of 1×THY supplemented with 1° A (w/v) glucose was added to each well. The plates were sealed to protect the specimens from contamination and incubated at 37° C. for 1 h to recharge the cells before measuring bioluminescence. Following a 1 h glucose boost, the bioluminescence of the specimens was measured at 590 nm in the Biotek Synergy HT system using a 1:2 luciferin (100 mM) to media volume ratio. Mean+standard deviation was obtained from five copolymer disks.

### Results:

### AMadh1 and AMadh2:

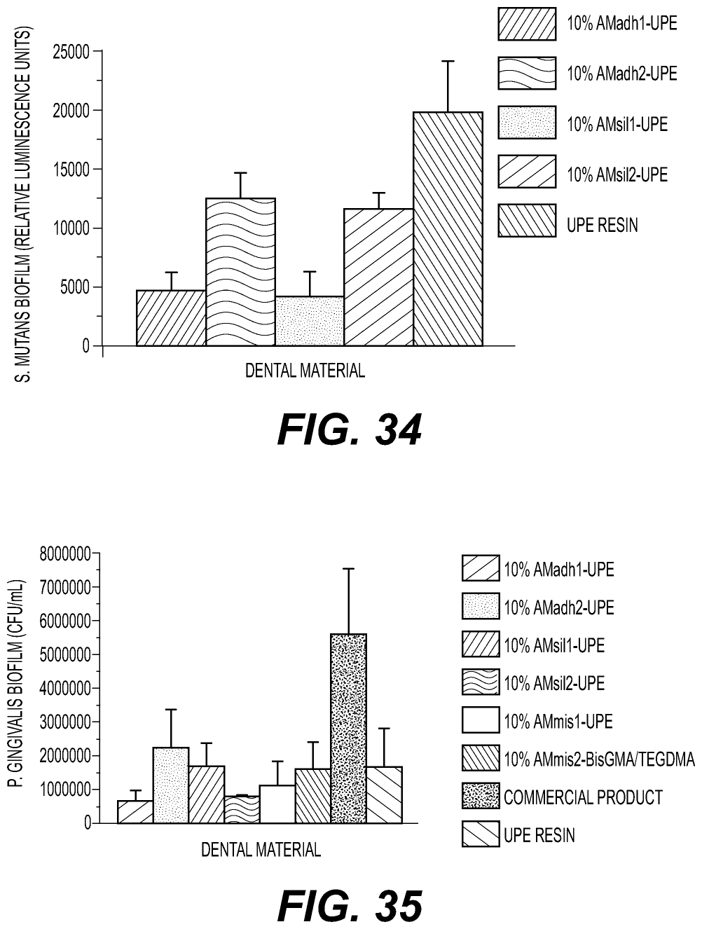

Compared to the UPE resin control, AMadh1-UPE and AMadh2-UPE (10% mass) copolymers reduced the colonization of S. mutans biofilm 4.2- and 1.6-fold, respectively (P≤0.006) (FIG. 34). FIG. 34 illustrates suppression of S. mutans biofilm growth by the experimental AMadh-UPE and AMsil-UPE copolymers compared to the UPE resin control. Indicated are mean value of relative luminescence units+standard deviation. S. mutans biofilms exposed to AMadh1-UPE were at least 2.5-fold lower (P≤0.006) than that observed with AMadh2-UPE.

### AMsil1 and AMsil2:

Compared to the UPE resin control, AMsil1-UPE and AMsil2-UPE (10% mass) copolymers reduced the colonization of S. mutans biofilm 4.7- and 1.7-fold, respectively (P≤0.002) (FIG. 34). S. mutans biofilms exposed to AMsil1-UPE were at least 2.8-fold lower (P≤0.005) than that observed with AMsil2-UPE.

Conclusion. Compared to the UPE control, all AMadh series monomers and AMsil series monomers exerted antimicrobial functionality (P≤0.006) against S. mutans biofilms. The dental monomers with shorter chain length (AMadh1 and AMsil1) had a more marked effect on reducing biofilms than their respective counterpart with an increased alkyl chain length (AMadh2 and AMsil2, respectively).

Antimicrobial Effect of Copolymer Resins Containing AM Monomers Against Biofilm Forms of Porphyromonas gingivalis:

### Methods:

Porphyromonas gingivalis (P. gingivalis), strain FDC 381 (ATCC® BAA-1703), was propagated in Becton Dickensen BBL chopped meat carbohydrate, pre-reduced II broth using a shaking incubator (37° C., anaerobic conditions). Three-day cultures were diluted in broth to approximate 5×10⁶ CFU/ml. Copolymer disks, vertically supported in a 24-well plate, were immersed in 1.6 ml of the bacterial suspension. In anaerobic conditions, the plate was incubated at 37° C. for 4 days. The copolymer disks were washed thrice in sterile 0.89% NaCl solution. Thereafter, the biofilm was displaced from the copolymer disks by transferring them to a sterile glass tube containing 1 ml of saline, vortexed (1 min), sonicated (10 min), and vortexed (1 min). Each disk was visually examined to ensure that the biomass was removed. The resulting suspensions were used to make ten-fold serial dilutions and subsequently spread onto the surface of Brucella agar with hemin and vitamin K1 (Sigma-Aldrich, St. Louis, Mo.) plates. After incubation (3 days at 37° C., anaerobic conditions), colony-forming units were enumerated. Mean+standard error was obtained from five copolymer disks tested independently.

### Results:

### AMadh1 and AMadh2:

Although not statistically different, P. gingivalis biomass on AMadh1-UPE disks was approximately 8.2 and 2.5-fold lower than that observed with a commercial antimicrobial dental material (containing methacryloyloxydodecyl pyrimidinium bromide) or the UPE resin control, respectively (FIG. 35). FIG. 35 illustrates suppression of P. gingivalis biofilm forms after exposure to 10% mass AMadh1-UPE, AMadh2-UPE, AMsil1-UPE, AMsil2-UPE, AMmis1-UPE, or AMmis2-BisGMA/TEGDMA copolymers. The commercial product was an antimicrobial dental restorative containing methacryloyloxydodecyl pyrimidinium bromide. The negative control group consisted of UPE resin. Bar height represents mean value of CFU/ml+standard error. P. gingivalis biomass on AMadh2-UPE disks was statistically comparable to that observed with the UPE resin; however, it was 2.5-fold lower than that observed with the commercial antimicrobial dental restorative.

### AMsil1 and AMsil2:

P. gingivalis biofilm biomass on copolymer disks exposed to AMsil1-UPE and AMsil2-UPE were lower (71% and 85%, respectively) than that observed with the commercial antimicrobial dental restorative (containing methacryloyloxydodecyl pyrimidinium bromide), albeit not statistically different (P=0.07) (FIG. 35).

### AMmis1 and AMmis2:

P. gingivalis biomass on AMmis1-UPE or AMmis2-BisGMA-TEGDMA disks were statistically comparable to that observed with the UPE resin (FIG. 35). However, when P. gingivalis biomass on AMₘᵢₛ₁-UPE or AMₘᵢₛ₂-BisGMA-TEGDMA disks were compared to the commercial antimicrobial dental restorative (containing methacryloyloxydodecyl pyrimidinium bromide), it was reduced nearly 5.0 and 3.5-fold, respectively.

Conclusion. Although not statistically different, the growth of P. gingivalis biofilms on the antimicrobial commercial restorative (containing methacryloyloxydodecyl pyrimidinium bromide) was notably greater than that observed in any of our experimental antimicrobial co-polymer resins (AMadh1-UPE, AMadh2-UPE, AMsil1-UPE, AMsil2-UPE, AMmis1-UPE, AMmis2-BisGMA-TEGDMA, and UPE resin control only).

### Experimental Series 4: Biocompatibility Testing of AM Monomers

### Methods:

### AMadh Series and AMsil Series Monomers:

Immortalized mouse subcutaneous connective tissue fibroblasts (NCTC clone 929 [L-cell, L-929, Strain L derivative]) were obtained from American Type Culture Collection, Manassas, Va.) (hereafter CCL1). Immortalized human gingival fibroblasts (HGF) were purchased from Applied Biological Materials, Inc. (Richmond, British Columbia, Canada). The cells were maintained (37° C., 5% CO₂) in 10% serum-supplemented Eagle's minimum essential medium and PriGrow III medium, respectively. For experiments, cells were obtained from a subconfluent stock culture.

Direct contact cytotoxicity testing was conducted as previously described^{7, 8}. Briefly, cells were exposed to two-fold serial dilutions of AMadh series monomers or AMsil series monomers to approximate the maximum possible exposure, (Table 9). Table 9 illustrates biocompatibility testing using maximum possible monomer exposure (mmol/L), assuming 7% mass fraction in the restorative with a maximum of 2% leaching. To account for multiplicity and variable size of restorations, a two- and four-fold greater dilution was also included in the testing. After 24 and 72 h, cells were assessed for cell viability using the LIVE/DEAD® Viability/Cytotoxicity kit (Life Technologies, Corp., Grand Island, N.Y.). Cellular metabolic activity was subsequently assessed using the CellTiter® AQueous One Solution Reagent (Promega, Corp., Madison, Wis.). Negative controls were without the novel monomer and/or cells. Mean+standard error was obtained from five independent replicates tested in triplicate.

TABLE 9
Biocompatability Test Results

| | Maximum | | Maximum | | Maximum |
|---|---|---|---|---|---|
| | possible | | possible | | possible |
| Monomer | exposure | | exposure (2-fold) | | exposure (4-fold) |
| AMadh1 | 5.3 | mmol/L | 10.6 | mmol/L | not done |
| AMadh2 | 4.4 | mmol/L | 8.8 | mmol/L | not done |
| AMsil1 | 4.17 | mmol/L | 8.34 | mmol/L | not done |
| AMsil2 | 3.64 | mmol/l | 7.28 | mmol/L | not done |
| AMmis1 | not done | | not done | | not done |
| AMmis2 | 7.25 | mmol/L | 14.49 | mmol/L | 28.98 mmol/L |

### AMmis2:

Immortalized human liver cells (hereafter HepG2-AD13) were obtained from collaborators at Aichi Gakuin University, Japan. The cells were maintained (37° C., 5% CO₂) in Dulbecco's Modified Eagle's medium supplemented with 10% fetal bovine serum. For experiments, cells were obtained from a subconfluent stock culture.

Direct contact cytotoxicity testing was conducted by exposing cells to two-fold serial dilutions of AMmis2 (Table 8) to approximate the maximum possible exposure, assuming 7% mass fraction in the composite with a maximum of 2% leaching. To account for multiplicity and variable size of restorations, a four-fold greater dilution was also included in the testing. After 6 h, cells were assessed for cell viability using a Luciferase Assay System (Promega Corp., Madison, Wis.). Negative controls were without the AMmis2 and/or cells. Mean+standard error was obtained from five independent replicates tested in quadruplicate.

### Results:

### AMadh1 and AMadh2:

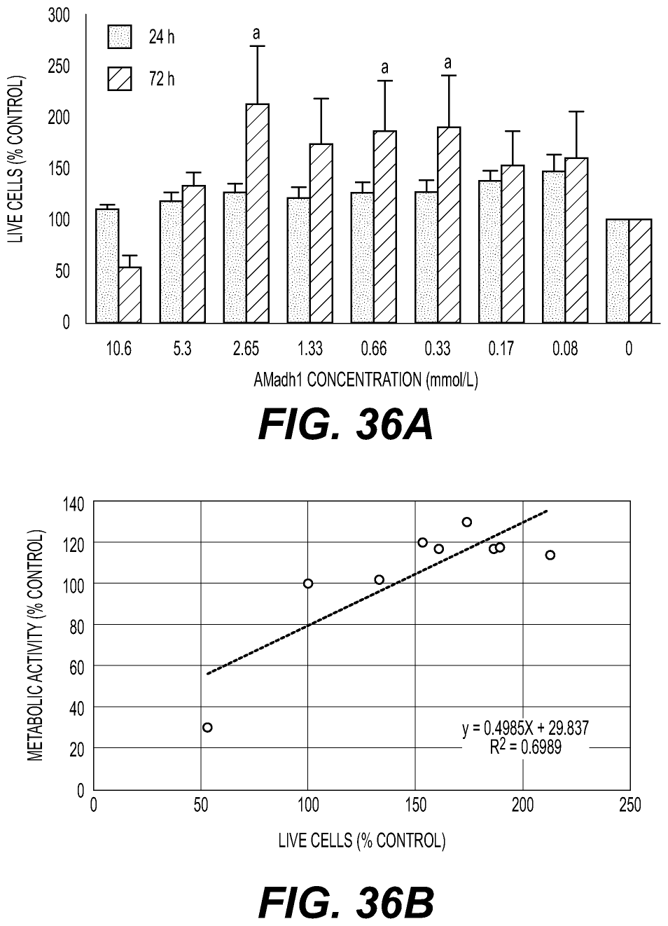

Both AMadh1 concentration and exposure time exerted a main effect (P≤0.04) on CCL1 cell viability (FIG. 36A). FIG. 36A illustrates the number of live CCL1 cells (% control) exposed to AMadh1 (≤10.6 mmol/L) for 24 h or 72 h. Bar height indicates mean value+standard error. Reference letter a in FIG. 36A indicates P≤0.05 when compared to 10.6 mmol/L AMadh1 within the same exposure time. Subsequent paired comparisons indicated that, at 24 h time exposure, the effect of monomer concentration was insignificant. After 72 h exposure to 10.6 mmol/L AMadh1, the mean number of live CCL1 cells decreased≤2.5-fold compared to all lower AMadh1 concentrations. However, paired comparisons indicated that the observed reduction was significant (P≤0.05) only at 2.65, 0.66, and 0.33 mmol/L AM_{adh1}. A main effect (P≤0.001) of AMadh1 concentration was also observed for the metabolic activity of CCL1 cells. Viability and metabolic activity of CCL1 cells exposed to AMadh1 for 72 h showed a positive linear correlation (R²=0.6989, P≤0.05; FIG. 36B). FIG. 36B illustrates the correlation (R²=0.6989) between the metabolic activity and viability of CCL1 cells exposed to AMadh1 for 72 h. The slope of the correlation line was calculated to be y=0.4985X+29.837.

In the AMadh1 series, monomer concentration affected (P≤0.02) the number of live HGF cells, regardless of time (Table 10). Table 10 illustrates the number (% control of live and dead cells) of human gingival fibroblasts exposed to AMadh1 (≤10.6 mmol/L) for 24 h or 72 h. The number of live cells in monomer-treated groups was greater (median of least square means=111.7%) than that observed in the control group. However, there were no significant paired comparisons within each exposure time. An effect (P≤0.03) of time was observed with HGF live cells (i.e., 10.4% difference of overall mean) exposed to AMadh1. Neither AMadh1 concentration nor time of exposure significantly affected the metabolic activity of HGF cells (data not shown).

TABLE 10
Effect of Monomer Concentration over Time

| | | |
|---|---|---|
| Amadh1 mmol/L | 24 h | 72 h |

| | | | |
|---|---|---|---|
| 10.6 | Live | 92.4 ± 10.8 | 103.7 ± 7.8 |
| | Dead | 100.9 ± 1.7 | 90.3 ± 11.6 |
| 5.3 | Live | 108.8 ± 14.8 | 130.5 ± 9.2 |
| | Dead | 102.7 ± 2.6 | 88.9 ± 6.8 |
| 2.65 | Live | 111.7 ± 10.0 | 107.8 ± 4.0 |
| | Dead | 100.4 ± 1.0 | 104.0 ± 17.1 |
| 1.33 | Live | 121.8 ± 8.2 | 107.6 ± 6.9 |
| | Dead | 110.9 ± 7.1 | 89.5 ± 8.9 |
| 0.66 | Live | 107.4 ± 4.7 | 133.0 ± 8.4 |
| | Dead | 101.5 ± 2.5 | 92.1 ± 8.7 |
| 0.33 | Live | 110.4 ± 9.7 | 116.8 ± 12.3 |
| | Dead | 103.5 ± 3.7 | 102.0 ± 12.8 |
| 0.17 | Live | 77.3 ± 15.9 | 95.1 ± 14.4 |
| | Dead | 99.1 ± 1.7 | 98.8 ± 9.1 |
| 0.08 | Live | 92.8 ± 11.3 | 121.6 ± 12.6 |
| | Dead | 102.1 ± 0.9 | 99.5 ± 8.0 |
| 0 | Live | 100 ± 0 | 100 ± 0 |
| | Dead | 100 ± 0 | 100 ± 0 |

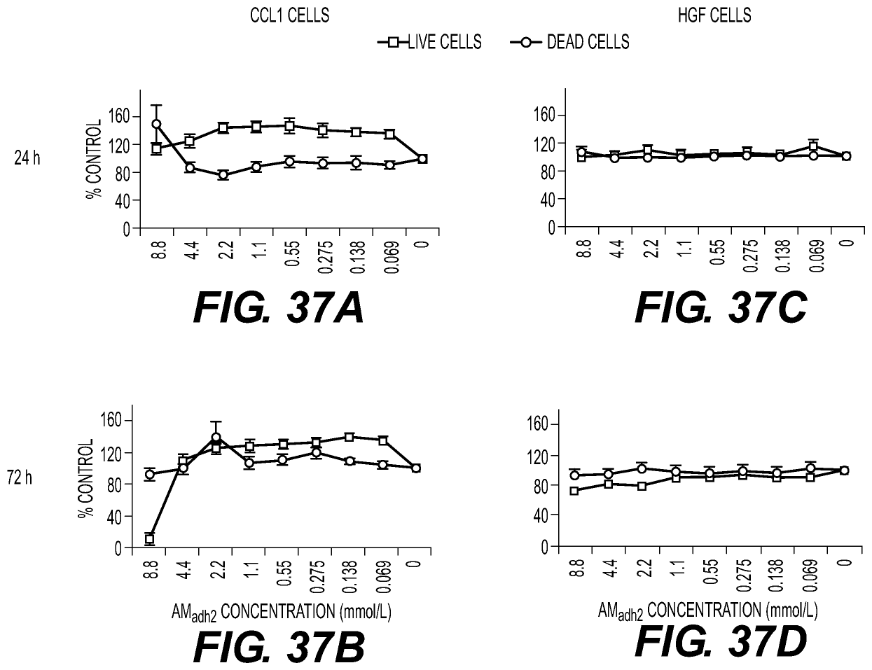

Independent of time, the least squares mean of live CCL1 cells exposed to 8.8 mmol/L AMadh2 was approx. 2-fold lower (P≤0.001) than that observed at lower concentrations (FIGS. 37A-D). FIGS. 37A-D illustrate the number (% control) of live and dead cells (left panels—CCL1 cells; right panels—HGFs) exposed to two-fold serial dilutions of AMadh2 (≤8.8 mmol/L) for 24 h (top panels) or 72 h (bottom panels). At 72 h exposure, the percentage of live CCL1 cells exposed to 8.8 mmol/L AMadh2 was at least 10-fold lower (P≤0.001) compared to AMadh2 concentrations≤4.4 mmol/L.

At concentrations of ≤2.2 mmol/L (24 h) and ≤0.28 mmol/L (72 h), the percent live CCL1 cells increased (P≤0.05) compared to control group (no monomer). Effect of AMadh2 concentration on the number of dead cells was not statistically different. Time of exposure had an effect (P≤0.001) on CCL1 cell viability. Like cell viability, both AM_{adh2} concentration and exposure time exerted an effect (P≤0.001) on CCL1 metabolic activity. At 24 h, exposure to 8.8 mm/L AMadh2 cell metabolic activity was reduced. Metabolic activity was markedly reduced at 72 h. Viability and metabolic activity of CCL1 cells exposed to AMadh2 showed a positive linear correlation at both 24 h (R²=0.40, P≤0.05) and 72 h (R²=0.87, P≤0.001) (data not shown).

AMadh2 concentration exhibited no significant effect on HGF cell viability, while time of exposure affected (P≤0.001) the percentage of live cells (FIGS. 37A-D). When comparing the effect of time, regardless of AMadh2 concentration, the number of live cells was consistently lower (˜16% difference of means). Paired comparisons between time points indicated that the effect was significant (P≤0.05) at 8.8, 2.2, and 0.07 mmol/L AMadh2. Analogous to viability, HGF metabolic activity was not affected by the level of AMadh2 in the resin. Time of exposure decreased cell metabolic activity (19.01 difference of means, P≤0.001). At 72 h, viability and metabolic activity of HGF cells exposed to AMadh2 showed a positive linear correlation (R²=0.60, P≤0.001) (data not shown).

Control wells containing no cells yielded a negligible optical density value. Control wells (with or without cells) in which the tetrazolium salt reagent was omitted, resulted in low optical density values. Positive control wells containing unexposed cells that were given an equal volume of culture medium were not significantly different from cells that were previously stained with the live-dead stain (data not shown).

### AMsil1 and AMsil2:

AMₛᵢₗ₁ concentration did not exert a statistical effect on the number (% control) of live and dead CCL1 cells (Table 11). Table 11 illustrates the number of live and dead CCL1 cells (% control) exposed to AMₛᵢₗ₁ (≤8.34 mmol/L) for 24 or 72 h. Exposure time reduced the number (1.2-fold difference of mean, P≤0.002) of live cells. Similarly, AMsil1 concentration did not affect the metabolic activity of CCL1 cells. Time of exposure had a modest effect (P≤0.01) on CCL1 metabolic activity, although no significant paired comparisons (within each concentration) were observed (data not shown).

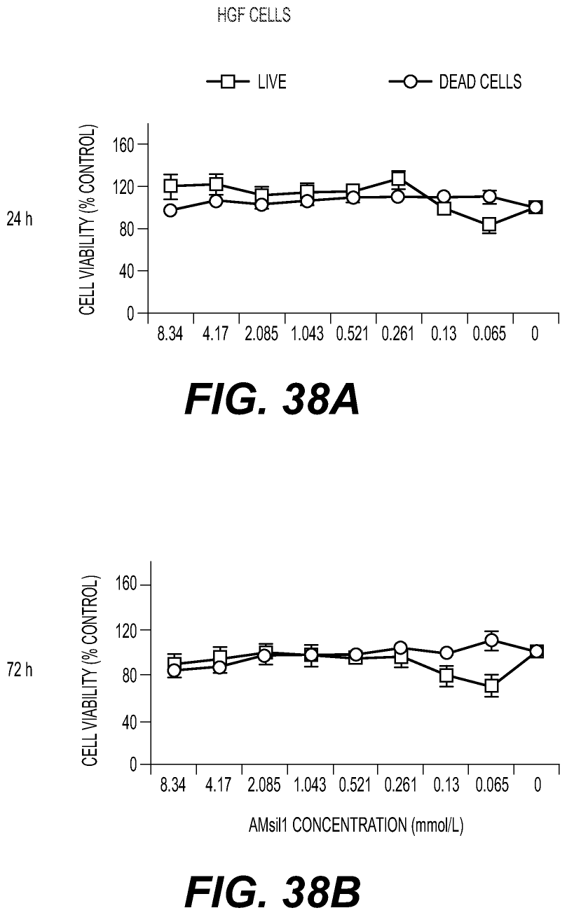

Independent of time, AMsil1 concentration exerted a main effect (P≤0.01) on the number of viable HGFs (FIGS. 38A and B). FIGS. 38A and B illustrate the number (% control) of live and dead HGF cells exposed to two-fold serial dilutions of AMsil1 (≤8.34 mmol/L) for 24 h (FIG. 38A) or 72 h (FIG. 38B). Paired comparisons indicated that at lower AMsil1 concentrations (≤0.13 mmol/L), the number of live HGFs decreased. Independent of AMsil1 concentration, exposure time reduced (19.73 difference of means, P≤0.001) HGF viability. Monomer concentration or time of exposure did not statistically affect the metabolic activity of HGF cells (data not shown).

TABLE 11
Number of Live and Dead CCL1 Cells
(% Control) Exposed to AMsil1

| AMsiL1 | | | |
|---|---|---|---|
| mmol/L | Stain | 24 h | 72 h |
| 8.34 | Live | 149.6 ± 37.4 | 110.8 ± 12.3 |
| | Dead | 93.0 ± 15.4 | 106.8 ± 7.4 |
| 4.17 | Live | 161.3 ± 15.2 | 132.1 ± 6.7 |
| | Dead | 85.8 ± 13.1 | 91.6 ± 13.7 |
| 2.09 | Live | 152.5 ± 20.3 | 136.0 ± 7.2 |
| | Dead | 86.1 ± 11.7 | 147.6 ± 38.5 |
| 1.04 | Live | 157.9 ± 18.8 | 142.0 ± 6.5 |
| | Dead | 96.0 ± 16.2 | 128.8 ± 20.2 |
| 0.52 | Live | 147.3 ± 20.3 | 122.9 ± 11.1 |
| | Dead | 117.4 ± 27.1 | 124.6 ± 22.9 |
| 0.26 | Live | 154.5 ± 25.0 | 108.9 ± 18.6 |
| | Dead | 96.8 ± 15.5 | 101,8 ± 10.8 |
| 0.13 | Live | 145.1 ± 14.8 | 122.1 ± 8.5 |
| | Dead | 85.1 ± 11.2 | 140.3 ± 32.7 |
| 0.07 | Live | 156.8 ± 21.4 | 122.5 ± 5.6 |
| | Dead | 128.0 ± 27.1 | 134.0 ± 36.5 |
| 0 | Live | 100 ± 0 | 100 ± 0 |
| | Dead | 100 ± 0 | 100 ± 0 |

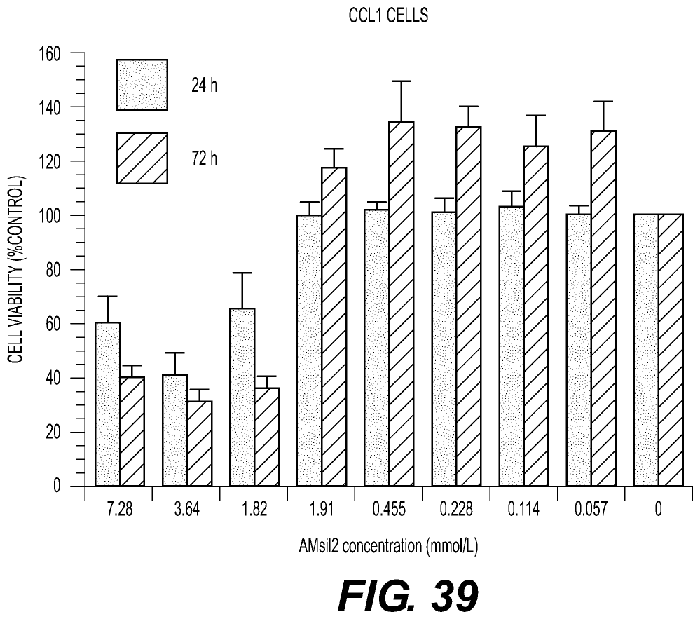

Regardless of time, AMsil2 concentration exerted a main effect (P≤0.001) on the number of viable CCL1s (FIG. 39). FIG. 39 illustrates the number of live CCL1 cells (% control) exposed to two-fold serial dilutions of AMsil2 (≤7.28 mmol/L) for 24 h or 72 h. Paired comparisons indicated that at 24 h exposure, CCL1 cells exposed to ≥3.64 mmol/L were lower (P≤0.05) than the number of live cells exposed to lower concentrations. At 72 h exposure, the percentage of live CCL1 cells exposed to ≥1.82 mmol/L AMsil2 was at least 3 to 4-fold lower (P≤0.001) compared to concentrations≤0.91 mmol/L. Like cell viability, AMsil2 concentration exerted an effect (P≤0.001) on CCL1 metabolic activity. Viability and metabolic activity of CCL1 cells exposed to AMsil2 showed a strong positive linear correlation at 24 h (R²=0.91, P≤0.005) and 72 h (R²=0.93, P≤0.0025) (data not shown).

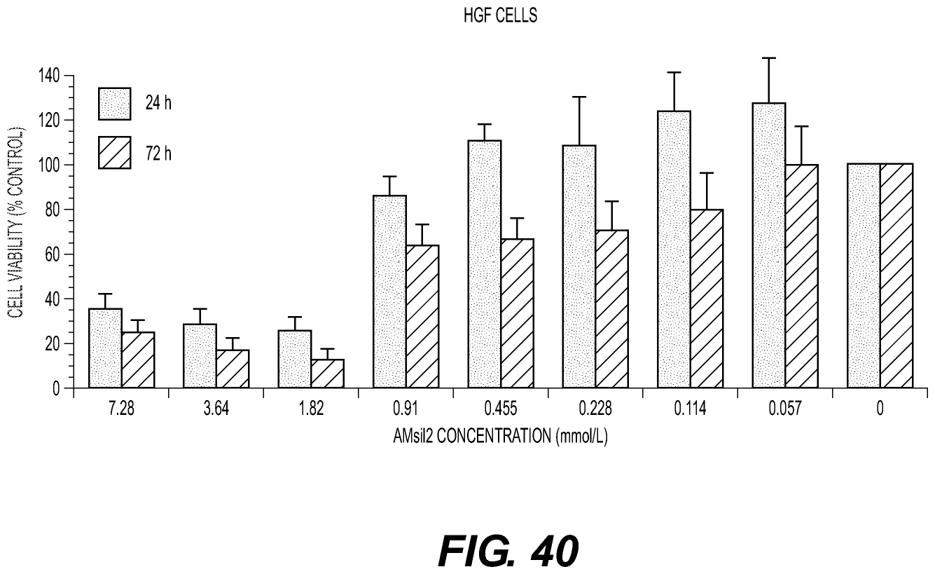

AMsil2 exhibited a concentration effect (P≤0.001) on HGF cell viability (FIG. 40). FIG. 40 illustrates the number (% control of live cells) of HGF cells exposed to two-fold serial dilutions of AMsil2 (≤7.28 mmol/L) for 24 h or 72 h. Paired comparisons indicated that exposure to ≥1.82 mmol/L AMsil2 reduced the number of live HGFs by more than 3-fold (P≤0.001), compared to the control group. When considering the effect of time, regardless of AMsil2 concentration, the number of live cells was consistently lower (˜23% difference of means). Although cell viability was decreased after 72 h with all AMsil2, significant comparisons (P≤0.05) between time were only observed at 0.455, 0.228, and 0.114 mmol/L. Like viability, AMsil2 concentration exhibited an effect (P≤0.001) on HGF metabolic activity. At 24 h and 72 h, viability and metabolic activity of HGF cells exposed to AMsil2 showed a positive linear correlation (R²=0.94 and R²=0.77, respectively) (data not shown).

Like that observed with AMadh series monomers, control wells containing no cells yielded a negligible optical density value. Control wells (with or without cells) in which the tetrazolium salt reagent was omitted, resulted in low optical density values. Positive control wells containing unexposed cells that were given an equal volume of culture medium were not significantly different from cells that were previously stained with the live-dead stain (data not shown).

### AMmis2:

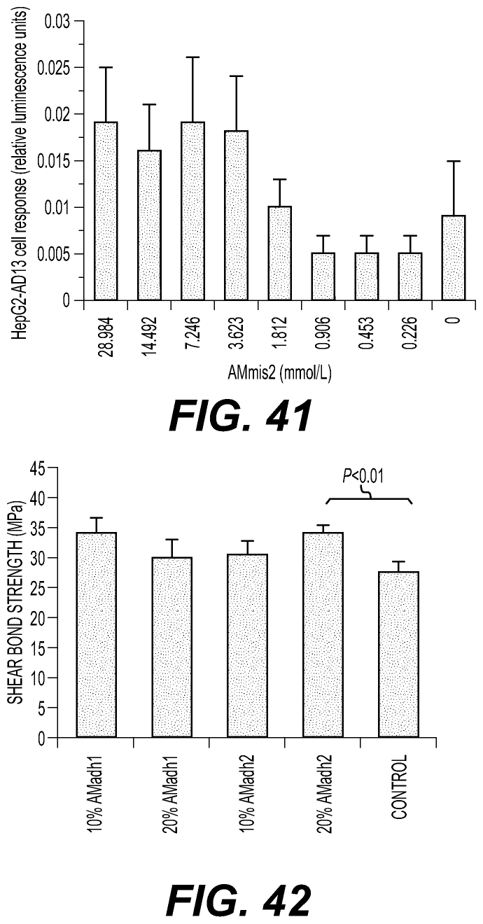

Compared to the negative control (no monomer), concentrations≤1.812 mmol/L of AMmis2 did not exert a significant effect on HepG2-AD13 cells (FIG. 41). FIG. 41 illustrates the relative luminescence units of HepG2-AD cells exposed to two-fold serial dilutions of AMmis2 (≤28.98 mmol/L) for 6 h. Bar height represents mean+standard error. Relative luminescence units observed at concentrations≥3.623 mmol/L were at least 1.5-fold greater than that observed at all lower concentrations.

Conclusion. The cytotoxic potential of novel dental monomers was evaluated in this study at concentrations representing accelerated leaching as found in UDMA-based resins⁹. HGFs were generally less sensitive to AMadh series and AMsil series monomer concentrations than CCL1 cells. The effect of exposure time on cells was contingent on the antimicrobial monomer type. Altogether, we deem these monomers (AMadh1, AMadh2, AMsil1, AMsil2, and AMmis2) as suitable AM candidates, as our experiments approximated the maximum possible exposure, which may be released over the service life of the restorative material.

### Experimental Series 5: Effect of AMadh Series Monomers on Shear Bond Strength (SBS) of a Multipurpose Dental Restorative Material

### Methods:

Extracted non-carious human molars were stored, sectioned, mounted, and polished as described.¹⁰ The use of discarded teeth to test SBS of dental resin adhesives was not considered human subject research and therefore did not require review and approval by the ADA Institutional Review Board (Assurance identification number 00009495). The bonding protocol was conducted at ambient conditions (74.5° F., 44% relative humidity). Briefly, the dentin was etched with 37% phosphoric acid. Each tooth was then rinsed with water (20 s) and blotted dry. Adper™ Scotchbond™ Multipurpose Primer (3M™ ESPE™ Co., St. Paul, Minn.), combined with AMadh1 or AMadh2 (10% or 20% wt), was applied for 20 s. Ensuring uniform air pressure, velocity, relative humidity (10.1%), and distance, the specimens were dried for 20 s. The Adper™ Scotchbond™ Multipurpose Primer, combined with AMadh1 or AMadh2 (10% or 20% wt) was applied again for 20 s. Immediately thereafter, Adper™ Scotchbond™ Multi-purpose Adhesive (3M™ ESPE™ Co.) was applied with a micro-brush for 10 s, followed by 20 s visible light irradiation. Subsequent application of the TPH Spectra Universal composite (Dentsply Sirona, Charlotte, N.C., USA) was confined by a Teflon-coated iris (approx. 12.3 mm circular area with 1.5 mm thickness). After assembly, the specimens were light-cured for 30 s. Negative control samples were processed likewise, except for the omission of the AMadh monomer from the primer. SBS measurements were performed with a Universal Testing Machine (United Calibration Corp., Huntington Beach, Calif., USA) with the knife-edge chisel engaging the bonding iris. Each experimental group consisted of a minimum of 10 teeth.

### Results:

Increasing AMadh1 concentrations (10% to 20%) in a commercial primer/adhesive system did not statistically affect the SBS (34.0±8.4 and 29.9±9.8, respectively) (FIG. 42). FIG. 42 illustrates shear bond strength of AMadh series monomers (10% or 20% wt) combined with Adper™ Scotchbond™ Multipurpose Primer to dentin/composite bonds. Bar height indicates mean value+standard deviation. Bracket indicates statistical difference (P<0.01) between 20% AMadh2 and commercial control. SBS of samples containing 10% or 20% AMadh2 had a slight dose response, although not statistically different (30.4±7.6 and 34.1±4.1, respectively). Although all groups containing AMadh series monomers had an increased SBS than that observed in the control, the only significant increase (P≤0.01) was observed with 20% AMadh2.

Conclusion. Shear bond strength of AMadh1 (10% or 20% wt) and AMadh2 (10% wt) was comparable to the commercial control. Incorporation of AMadh2 (20% wt) increased (P<0.01) the SBS over that observed in the UPE resin control. The SBS of AMadh1 (range 29.9-34.0 MPa) and AMadh2 (range 30.4-34.1) to dentin were well above that reported with other adhesive systems^{11, 12}. Moreover, SBS of AMadh series monomers are well within the ranges reported in a systematic review/meta-analysis of dentin bond strength of universal adhesives (etch-and-rinse (range 16.8-54.6 MPa, mean=37.4) and self-etch (range 11.5-54.4 MPa, mean=32.6))¹³.

### REFERENCES FOR EXPERIMENTAL SERIES 3-5

1. Bienek D R, Giuseppetti A A and Skrtic D. Amorphous calcium phosphates as bioactive filler in polymeric dental composites. In: Dutour-Sikiric M and Furedi-Milhofer H (eds) Calcium Phosphates—From Fundamentals to Applications. London, UK: IntechOpen Limited, 2019.
2. Goy R C, Britto D and Assis O B G. A review of the antimicrobial activity of chitosan. Polimeros: Ciência e Tecnologia 2009; 19: 241-247.
3. Bienek D R, Hoffman K M and Tutak W. Blow-spun chitosan/PEG/PLGA nanofibers as a novel tissue engineering scaffold with antibacterial properties. J Mater Sci: Mater Med 2016; 27: 146. 2016 Aug. 29. DOI: 10.1007/s10856-016-5757-7.
4. Merritt J, Kreth J, Qi F, et al. Non-disruptive, real-time analyses of the metabolic status and viability of Streptococcus mutans cells in response to antimicrobial treatments. J Microbiol Methods 2005; 61: 161-170. 2005 Feb. 22. DOI: 10.1016/j.mimet.2004.11.012.
5. Bienek D R, Giuseppetti A A, Okeke U C, et al. Antimicrobial, biocompatibility, and physicochemical properties of novel adhesive methacrylate dental monomers. J Bioact Compat Polym 2019 (submitted).
6. Esteban Florez F L, Fliers R D, Smart K, et al. Real-time assessment of Streptococcus mutans biofilm metabolism on resin composite. Dent Mater 2016; 32: 1263-1269. 2016 Aug. 16. DOI: 10.1016/j.dental.2016.07.010.
7. Bienek D R, Frukhtbeyn S A, Giuseppetti A A, et al. Ionic dimethacrylates for antimicrobial and remineralizing dental composites. Annals of Dentistry and Oral Disorders 2018; 1: 108.
8. Bienek D R, Frukhtbeyn S A, Giuseppetti A A, et al. Antimicrobial monomers for polymeric dental restoratives: Cytotoxicity and physicochemical properties. J Funct Biomat 2018; 9: 20. DOI: 10.3390/jfb9010020.
9. Skrtic D and Antonucci J M. Bioactive polymeric composites for tooth mineral regeneration: Physicochemical and cellular aspects. J Funct Biomat 2011; 2: 271-307. 2011 Nov. 22. DOI: 10.3390/jfb2030271.
10. Schumacher G E, Eichmiller F C and Antonucci J M. Effects of surface-active resins on dentin/composite bonds. Dent Mater 1992; 8: 278-282. 1992 Jul. 1.
11. Mortazavi V, Fathi M, Ataei E, et al. Shear bond strengths and morphological evaluation of filled and unfilled adhesive interfaces to enamel and dentine. Int J Dent 2012; 2012: 858459. 2012 Dec. 5. DOI: 10.1155/2012/858459.
12. Yazici A R, Celik C, Ozgunaltay G, et al. Bond strength of different adhesive systems to dental hard tissues. Oper Dent 2007; 32: 166-172. 2007 Apr. 13. DOI: 10.2341/06-49.
13. Rosa W L, Piva E and Silva A F. Bond strength of universal adhesives: A systematic review and meta-analysis. J Dent 2015; 43: 765-776. 2015 Apr. 18. DOI: 10.1016/j.jdent.2015.04.003.

We claim:

1. A polymerizable multifunctional antimicrobial monomer, comprising:
a quaternary ammonium cation group, wherein the quaternary ammonium cation group is bonded to functional groups and anions having structures chosen from the group consisting of: 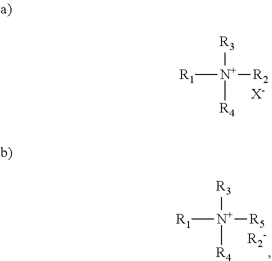
wherein:
structure a) comprises functional groups R₁, R₂, R₃, R₄ and anion X; and
structure b) comprises functional groups R₁, R₃, R₄, R₅ and anion R₂, wherein:
R₁ contains an alkenyl group,
structure a) is one of an ammonium monomer adhesive (AMadh) compound and an ammonium monomer silane (AMsil) compound, and
structure b) is an ammonium monomer miscible (AMmis) compound.

2. The composition as recited in claim 1, wherein R₁ contains vinyl groups.

3. The composition as recited in claim 1, wherein R₁ contains acrylate groups.

4. The composition as recited in claim 1, wherein R₁ contains methacrylate groups.

5. The composition as recited in claim 1, wherein R₂ includes at least one of a carboxylic, organo-silane, organo-sulfate, ionic acrylate or ionic methacrylate groups.

6. The composition as recited in claim 1, wherein for the AMadh series compounds,
R₁ comprises an alkenyl group that functions as polymerization site between quaternary ammonium monomers and copolymer or composite resins;
the alkenyl group is chosen from the group consisting of vinyl (CH₂═CH—), methacrylic (CH₂═C(CH₃)—) and crotyl (CH₃—CH═CH—CH₂—) groups;
the R₂ group comprises a carboxylic group; and
the anion X is a halogen anion (F, Cl, Br, I).

7. The composition of claim 6, wherein the AMadh series compounds interact with the copolymer resin matrix and tooth enamel and dentin structures through its added adhesive functionalities.

8. The composition as recited in claim 1, wherein for the AMsil series compounds,
R₁ comprises an alkenyl group that functions as a polymerization site between quaternary ammonium monomers and copolymer or composite resins;
the alkenyl group is chosen from the group consisting of vinyl (CH₂═CH—), methacrylic (CH₂═C(CH₃)—) and crotyl (CH₃—CH═CH—CH₂—) groups;
the R₂ group comprises an organo-silane group which functions as a coupling agent to fillers and resin polymer matrix; and
the anion X is a halogen anion (F, Cl, Br, I).

9. The composition of claim 8, wherein the AMsil compounds couple with both filler phase and polymer phase of the composites.

10. The composition as recited in claim 1, wherein for AMmis1 compounds:
R₁ comprises an alkenyl group that functions as a polymerization site between quaternary ammonium monomers and copolymer or composite resins;
the alkenyl group is chosen from the group consisting of vinyl (CH₂═CH—), methacrylic (CH₂═C(CH₃)—) and crotyl (CH₃—CH═CH—CH₂—) groups; and
the anion R₂ comprises an organo-sulfate group with a carbon tail.

11. The composition as recited in claim 1, wherein for AMmis2 compounds:
R₁ comprises an alkenyl group that functions as polymerization site between quaternary ammonium monomers and copolymer or composite resins;
the alkenyl groups are chosen from the group consisting of vinyl (CH₂═CH—), methacrylic (CH₂═C(CH₃)—) and crotyl (CH₃—CH═CH—CH₂—) groups; and
the anion R₂ comprises an ionic acrylate or methacrylate functionality.

12. A polymerizable composition comprising one of a liquid copolymer and a liquid composite resin, the liquid copolymer and the liquid composite resin comprising initiator(s), reinforcing or remineralizing fillers, and antimicrobial monomers, the antimicrobial monomers containing a quaternary ammonium group bonded to respective groups R₁, R₂, R₃, R₄ and R₅ such that, when initiated, the liquid copolymer and the liquid composite resin comprising the antimicrobial monomers polymerize to form a solid copolymer or a solid composite resin.

13. The polymerizable composition as recited in claim 12, wherein the polymerizable composition includes up to 50 wt. % of the antimicrobial monomers.

14. The polymerizable composition as recited in claim 12, wherein the liquid copolymer or the liquid composite resin is selected from a group consisting of triethylene glycol dimethacrylate (TEGDMA), hydroxyethyl methacrylate (HEMA), ethyl hydroxymethyl acrylate (EHMA), bisphenol A-glycidyl methacrylate (bisGMA), urethane dimethacrylate (UDMA), polyethylene glycol extended urethane dimethacrylate (PEG-UDMA), and mixtures thereof.

15. The polymerizable composition as recited in claim 14, wherein the polymerizable composition includes an amount X_{(diluent monomers)}, wt. % of the TEGDMA, HEMA, EHMA and an amount X_{(base monomers)} wt. % of the bis-GMA, UDMA, PEG-UDMA, wherein X_{(diluent monomers)} and X_{(base monomers)} are each 10-90 wt. % and X_{(diluent monomers)}+X_{(base monomers)} equals 100%.

16. The polymerizable composition as recited in claim 14, further comprising one or more initiators chosen from the group consisting of camphorquinone (CQ) and ethyl 4-N,N-dimethylaminobenzoate (4E).

17. The polymerizable composition as recited in claim 14, wherein the solid copolymer or solid composite resin exert an antimicrobial effect on planktonic and biofilm forms of microorganisms.

18. A polymerizable multifunctional antimicrobial composition, comprising:
a base monomer;
a diluent monomer; and
a quaternary ammonium cation group, wherein the quaternary ammonium cation group is bonded to functional groups and anions having structures chosen from the group consisting of: 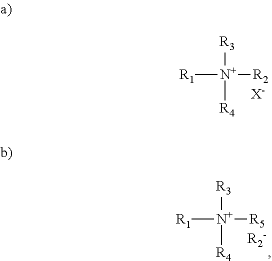
wherein:
structure a) comprises functional groups R₁, R₂, R₃, R₄ and anion X; and
structure b) comprises functional groups R₁, R₃, R₄, R₅ and anion R₂, wherein:
R₁ contains an alkenyl group,
structure a) is one of an ammonium monomer adhesive (AMadh) compound and an ammonium monomer silane (AMsil) compound, and
structure b) is an ammonium monomer miscible (AMmis) compound.

19. The polymerizable multifunctional antimicrobial composition of claim 18, wherein a solid copolymer or solid composite resin formed from the polymerizable multifunctional antimicrobial composition exert an antimicrobial effect on planktonic forms of Streptococcus mutans.

20. The polymerizable multifunctional antimicrobial composition of claim 18, wherein a solid copolymer or solid composite resin formed from the polymerizable multifunctional antimicrobial composition exert an antimicrobial effect on biofilm forms of Streptococcus mutans.

21. The polymerizable multifunctional antimicrobial composition of claim 18 wherein a solid copolymer or solid composite resin formed from the polymerizable multifunctional antimicrobial composition exert an antimicrobial effect on biofilm forms of Porphyromonas gingivalis.
